(19) United States
(12) Patent Application Publication
(10) Pub. No.: US 2020/0173997 A1
(43) Pub. Date: Jun. 4, 2020

(54) CHOLESTERYL LINOLEATE (18:2) IN FECES SAMPLES AS BIOMARKER FOR COLORECTAL CANCER

(71) Applicants: SERVIZO GALEGO DE SAÚDE (SERGAS), Santiago de Compostela (ES); ASOCIACIÓN CENTRO DE INVESTIGACIÓN COOPERATIVA EN BIOCIENCIAS - CIC BIOGUNE, Derio (ES); ADMINISTRACIÓN GENERAL DE LA COMUNIDAD AUTÓNOMA DE EUSKADI, Vitoria-Gasteiz (ES); CENTRO DE INVESTIGACIÓN BIOMÉDICA EN RED, Madrid (ES)

(72) Inventors: Joaquín CUBIELLA FERNÁNDEZ, Ourense (ES); Juan Manuel FALCÓN PÉREZ, Derio (ES); Luis BUJANDA FERNÁNDEZ DE PIEROLA, Donstia/ San Sebastian (ES)

(21) Appl. No.: 16/612,343

(22) PCT Filed: May 11, 2018
(86) PCT No.: PCT/EP2018/062257

§ 371 Date: Nov. 8, 2019

(30) Foreign Application Priority Data
May 11, 2017 (EP) 17382267.7

(51) Int. Cl. G01N 33/574; G01N 33/92; G16H 50/70

(52) U.S. Cl. CPC G01N 33/57419; G16H 50/70; G01N 33/92

(57) ABSTRACT

The present invention relates to biomarkers for colorectal cancer. Specifically, it relates to metabolic markers as a screening, diagnostic or monitoring tool for detecting colorectal cancer (CRC) and/or advanced adenoma (AD) in a subject. In a particular embodiment, it refers to an in vitro method for the screening, diagnosis or monitoring of colorectal cancer and/or advanced adenoma in a subject, said method comprising the following steps: a) determining the levels of the metabolic marker ChoE(18:2) in a feces sample isolated from said subject; b) comparing the levels in said feces sample with a reference value; wherein an increase of ChoE(18:2) levels in the subject sample with regard to said reference value is indicative of CRC and/or AD.

### FIELD OF THE INVENTION

The present invention relates to biomarkers for colorectal cancer. Specifically, it relates to metabolic markers as a screening, diagnostic or monitoring tool for detecting colorectal cancer (CRC) and/or advanced adenoma (AD) in a subject. In particular, it refers to ChoE(18:2) alone or in combination with other biomarkers, such as fecal occult blood or at least one further metabolic marker selected from the group consisting of ChoE(18:0), SM(d18:0/14:0), TG(54:3), ChoE(18:1), ChoE(20:4), PE(16:0/18:1), SM(d18:1/23:0), SM(42:3) and TG(54:1).

### BACKGROUND OF THE INVENTION

Colorectal cancer (CRC) is the second leading cause of cancer death in developed countries (Int J Cancer, 2012, 136, E359-386). Although the knowledge of the genetics- and diet-associated mechanisms involved in CRC detection and progression is increasing rapidly (Science, 2013, 339, 1546-1558) still the best prognosis is obtained when malignancy is early detected. CRC screening, which detects both precancerous polyps and CRC, can reduce both colorectal cancer incidence and mortality (Gastroenterology, 2004, 126, 1674-1680; Br J Surg, 2008, 95, 1029-1036; N Engl J Med, 2012 366, 697-706; Lancet, 2010, 375, 1624-1633; N Engl J Med, 2012 366, 687-696). Through screening, the incidence of colorectal cancer can be reduced by 30% with a mortality reduction of 50% depending on the screening modality and participation rate (Lancet, 2010, 375, 1624-1633; J Natl Cancer Inst, 2005, 97, 347-357). These data provide support for having efficient and sensitive screening methods.

Present screening tests available include examining stool for occult blood (N Engl J Med, 2012, 366, 697-706) or tests examining DNA mutations/alterations (N Engl J Med, 2014, 370, 1287-1297), radiologic or endoscopic (flexible sigmoidoscopy, colonoscopy, and computed tomographic colonography) methods (Gastroenterology, 2008, 134, 1570-1595). Among the available screening tests, colonoscopy is considered to be the most accurate test for early detection and prevention of colorectal cancer (N Engl J Med, 2006, 355, 1863-1872). However, colonoscopy has several limitations including some secondary effects mostly a high level of discomfort that causes a low level of adherence of average-risk and familial-risk colorectal cancer screenings (Gut, 2007, 56, 1714-1718; Colorectal Dis, 2011, 13, 1463-1318). Furthermore, it is a procedure requiring one or more days of dietary preparation and bowel cleansing and a day dedicated to the examination. In order to monitor the progression of the disease in symptomatic patients by routine testing, low-invasive methods have been developed (Dig Liver Dis, 2015, 47, 797-804).

Metabolomics refers to the comprehensive analysis of endogenous and xenobiotics small molecules of less than 2000 Da present in a biological system. As a result of the recent technological development of analytical instruments combined with the rapid progress in bioinformatics it is now possible to quickly and simultaneously measure and model large numbers of metabolites in biological samples (Drug Metab Rev, 2007, 39, 581-597; Toxicol Pathol, 2008, 36, 140-147; Nat Rev Mol Cell Biol, 2004, 5, 763-769; Nat Rev Drug Discov, 2003, 2, 668-676). Consequently, metabolomics has found broad application in the identification of biomarkers, and in the unraveling of pathophysiological mechanisms in many scientific fields including cancer. The development of ultra-performance liquid chromatography (UPLC) has made it possible to achieve even higher resolution, higher sensitivity, and rapid separation when compared to conventional high-performance liquid chromatography (HPLC) methods (Anal Chem, 2006, 78, 3289-3295; J Sep Sei, 2006, 29, 2433-2443).

Metabolomic study of feces may be more effective in detecting novel colon cancer makers than other approaches because feces are in close proximity to the colorectal mucosa and is a product of interaction between dietary components and microbiota. Microbiota is affected by and seems to play an important role in the progression of colon cancer (Mol Cell, 2014, 54, 309-320; Front Microbiol, 2015, vol. 6, article 20).

It has been reported that dyslipidemia is an important component of metabolic syndrome and may contribute to colorectal carcinogenesis through insulin resistance, oxidative stress, and inflammatory pathways (Cowey S, Hardy R W. Am J Pathol 2006; 169: 1505-22; Van Duijnhoven F J et al. Gut 2011; 60:1094-102). Other studies have reported that hypertriglyceridemia, high total cholesterol levels, low-density lipoprotein (LDL) cholesterol levels, and high-density lipoprotein (HDL) cholesterol levels were associated with increased risk of colorectal adenomas. However, most of these studies included a relatively small number of subjects and the associations were inconsistent (Yang M H et al., Am J Gastroenterol 2013; 108:833-841).

Tosi M R and Tugnoli V. (Clin Chim Acta. 2005; 359(1-2):27-45) generally describe an association between intracellular cholesteryl esters (ChoE) and cancer. Specifically, it refers to Gebhard et al. (J Lipid Res. 1987 October; 28(10):1177-84) which described for renal carcinoma an increase of 35× of ChoE in malignant cells with respect to the healthy kidney. Tosi and Tugnoli proposed that ChoE is formed intracellularly considering that oleate (18:1) is the form predominant whereas linoleate (18:2) is the form predominant in circulating lipoproteins. In table 1 of Gebhard et al. is shown the lipid fraction of ChoE extracted from healthy and cancer kidney cells, respectively. It was reported that there was an increase of oleate (18:1) in cancer cells whereas the levels of linoleate (18:2) are higher in healthy kidney cells. This is indeed the opposite of what has been found by the authors of the present invention for colorectal cancer where the levels of linoleate (18:2) in fecal samples are found to be increased in those patients suffering from colorectal cancer.

Despite the advances in the field of colorectal cancer detection, there still exists a need for the development of simple, cost-effective, low invasive, high sensitivity test for the screening, diagnosis and/or monitoring of colorectal cancer, as well as for monitoring of therapy efficacy.

### SUMMARY OF THE INVENTION

The authors of the present invention have identified a series of metabolic markers present in feces samples collected from subjects previously diagnosed with colorectal cancer (CRC) or advanced adenoma (AD). The selected metabolic markers are significantly differentiated between Healthy Controls (HC) and CRC or advanced adenoma patients, and/or between CRC and advanced adenoma patients. These metabolic markers can thus be used in a non-invasive screening, diagnostic or monitoring method identifying and classifying patients with CRC and/or advanced adenoma.

A particularly relevant biomarker has been found to be ChoE(18:2) which enables to discriminate with an AUC of 0.688 between HC and subjects having CRC and/or advanced adenoma (AD). Moreover, this biomarker has proven to be particularly good in discriminating between HC and subjects with CRC (with an AUC of 0.812). In addition, ChoE(18:2) may further be used for the differential diagnosis between CRC and AD where it has shown to have a discrimination power between these two population groups corresponding to an AUC of 0.766.

Accordingly, in a first aspect, the present invention provides an in vitro non-invasive method for the screening, diagnosis or monitoring of colorectal cancer and/or advanced adenoma in a subject, comprising determining in a biological sample of said subject the levels of at least one metabolic marker selected from the group consisting of ChoE(18:2), ChoE(18:0), ChoE(18:1), ChoE(20:4), PE(16:0/18:1), SM(d18:0/14:0), SM(d18:1/23:0), SM(42:3), TG(54:3) and TG(54:1), as described in Table 1, and comparing the levels of said marker or markers with respect to the levels of the same marker or markers in a HC patient or with respect to the same marker or markers in an CRC or in an advanced adenoma patient or with respect to a reference value as described through-out the present specification, wherein the subject is classified as having CRC and/or advanced adenoma respectively as explained throughout the specification and in the examples.

Preferred biomarkers are selected from the group consisting of ChoE(18:2), ChoE(18:1), ChoE(20:4), PE(16:0/18:1), SM(d18:1/23:0), SM(42:3) and TG(54:1). This group of biomarkers, referred herein as the “second signature”, was selected further to an iteration process designed to identify the most robust biomarkers and signature (i.e. the combined use of all these biomarkers) which retained the predictive character independently of the tested population sample. The ROC curve results for the control vs disease (CRC+AD), control vs CRC and CRC vs AD comparisons are provided in tables 6 and 7.

The method of the present invention can be used to diagnose colorectal cancer and/or advanced adenoma in individuals suspected of having advanced adenoma and/or colorectal cancer. It may also be used for the differential diagnosis between colorectal cancer and advanced adenoma.

The method of the present invention can also be used in the screening of colorectal cancer and/or advanced adenoma in individuals that do not present symptoms of intestinal disease (seemingly healthy individuals). Screening tests may be carried out in subjects with no personal or family history of colonic neoplasm, or other risk factors of CRC. Screening tests may also be conducted in individuals presenting an increased and/or high risk of colorectal cancer.

The present disclosure also provides a method of monitoring disease progression and/or therapy efficacy of colorectal cancer and/or advanced adenoma, for instance, to identify relapses in previously diagnosed patients after disease remittance.

The invention further relates to a method of treating a subject suffering from CRC and/or advanced adenoma, wherein said method comprises identifying the subject to be treated by a method comprising determining in a feces sample of said subject the levels of at least one metabolic marker selected from the group consisting of ChoE(18:2), ChoE(18:0), ChoE(18:1), ChoE(20:4), PE(16:0/18:1), SM(d18:0/14:0), SM(d18:1/23:0), SM(42:3), TG(54:3) and TG(54:1), as described herein, and treating said subject suffering from CRC and/or advanced adenoma.

As described herein below, the discrimination power of ChoE(18:2) may increase when combined with other biomarkers. Accordingly, methods which include the combined determination of ChoE(18:2) with other biomarkers, such as the other metabolic biomarkers described herein and/or the determination of presence of blood in feces (e.g. the FOB test), are also part of the methods of the invention.

### BRIEF DESCRIPTION OF THE DRAWINGS

FIG. 1. Receiver Operating Characteristic (ROC) curve for ChoE(18:0) alone (FIG. 1A), and in combination with the Fecal Occult Blood (FOB) test (FIG. 1B), for discrimination between control and disease (CRC+AD).

FIG. 2. ROC curve for ChoE(18:2) alone (FIG. 2A), and in combination with the FOB test (FIG. 2B), for discrimination between control and disease (CRC+AD).

FIG. 3. ROC curve for SM(d18:0/14:0) alone (FIG. 3A), and in combination with the FOB test (FIG. 3B), for discrimination between control and disease (CRC+AD).

FIG. 4. ROC curve for TG(54:3) alone (FIG. 4A), and in combination with the FOB test (FIG. 4B), for discrimination between control and disease (CRC+AD).

FIG. 5. ROC curve for the combined 4 metabolites (FIG. 5A), and in combination with the FOB test (FIG. 5B), for discrimination between control and disease (CRC+AD).

FIG. 6. ROC curve for ChoE(18:0) alone (FIG. 6A), and in combination with the FOB test (FIG. 6B), for discrimination between control and CRC.

FIG. 7. ROC curve for ChoE(18:2) alone (FIG. 7A), and in combination with the FOB test (FIG. 7B), for discrimination between control and CRC.

FIG. 8. ROC curve for SM(d18:0/14:0) alone (FIG. 8A), and in combination with the FOB test (FIG. 8B), for discrimination between control and CRC.

FIG. 9. ROC curve for TG(54:3) alone (FIG. 9A), and in combination with the FOB test (FIG. 9B), for discrimination between control and CRC.

FIG. 10. ROC curve for the combined 4 metabolites (FIG. 10A), and in combination with the FOB test (FIG. 10B), for discrimination between control and CRC.

FIG. 11. ROC curve for ChoE(18:0) alone (FIG. 11A), and in combination with the FOB test (FIG. 11B), for discrimination between CRC and AD.

FIG. 12. ROC curve for ChoE(18:2) alone (FIG. 12A), and in combination with the FOB test (FIG. 12B), for discrimination between CRC and AD.

FIG. 13. ROC curve for SM(d18:0/14:0) alone (FIG. 13A), and in combination with the FOB test (FIG. 13B), for discrimination between CRC and AD.

FIG. 14. ROC curve for TG(54:3) alone (FIG. 14A), and in combination with the FOB test (FIG. 14B), for discrimination between CRC and AD.

FIG. 15. ROC curve for the combined 4 metabolites (FIG. 15A), and in combination with the FOB test (FIG. 15B), for discrimination between CRC and AD.

FIG. 16. ROC curve for ChoE(18:2) alone (FIG. 16A), and in combination with the FOB test (FIG. 16B), for discrimination between control and disease (CRC+AD).

FIG. 17. ROC curve for ChoE(18:1) alone (FIG. 17A), and in combination with the FOB test (FIG. 17B), for discrimination between control and disease (CRC+AD).

FIG. 18. ROC curve for ChoE(20:4) alone (FIG. 18A), and in combination with the FOB test (FIG. 18B), for discrimination between control and disease (CRC+AD).

FIG. 19. ROC curve for PE(16:0/18:1) alone (FIG. 19A), and in combination with the FOB test (FIG. 19B), for discrimination between control and disease (CRC+AD).

FIG. 20. ROC curve for SM(d18:1/23:0) alone (FIG. 20A), and in combination with the FOB test (FIG. 20B), for discrimination between control and disease (CRC+AD).

FIG. 21. ROC curve for SM(42:3) alone (FIG. 21A), and in combination with the FOB test (FIG. 21B), for discrimination between control and disease (CRC+AD).

FIG. 22. ROC curve for TG(54:1) alone (FIG. 22A), and in combination with the FOB test (FIG. 22B), for discrimination between control and disease (CRC+AD).

FIG. 23. ROC curve for the combined 7 metabolites (FIG. 23A), and in combination with the FOB test (FIG. 23B), for discrimination between control and disease (CRC+AD).

FIG. 24. ROC curve for ChoE(18:2) alone (FIG. 24A), and in combination with the FOB test (FIG. 24B), for discrimination between control and CRC.

FIG. 25. ROC curve for ChoE(18:1) alone (FIG. 25A), and in combination with the FOB test (FIG. 25B), for discrimination between control and CRC.

FIG. 26. ROC curve for ChoE(20:4) alone (FIG. 26A), and in combination with the FOB test (FIG. 26B), for discrimination between control and CRC.

FIG. 27. ROC curve for PE(16:0/18:1) alone (FIG. 27A), and in combination with the FOB test (FIG. 27B), for discrimination between control and CRC.

FIG. 28. ROC curve for SM(d18:1/23:0) alone (FIG. 28A), and in combination with the FOB test (FIG. 28B), for discrimination between control and CRC.

FIG. 29. ROC curve for SM(42:3) alone (FIG. 29A), and in combination with the FOB test (FIG. 29B), for discrimination between control and CRC.

FIG. 30. ROC curve for TG(54:1) alone (FIG. 30A), and in combination with the FOB test (FIG. 30B), for discrimination between control and CRC.

FIG. 31. ROC curve for the combined 7 metabolites (FIG. 31A), and in combination with the FOB test (FIG. 31B), for discrimination between control and CRC.

FIG. 32. ROC curve for ChoE(18:2) alone (FIG. 32A), and in combination with the FOB test (FIG. 32B), for discrimination between CRC and AD.

FIG. 33. ROC curve for ChoE(18:1) alone (FIG. 33A), and in combination with the FOB test (FIG. 33B), for discrimination between CRC and AD.

FIG. 34. ROC curve for ChoE(20:4) alone (FIG. 34A), and in combination with the FOB test (FIG. 34B), for discrimination between CRC and AD.

FIG. 35. ROC curve for PE(16:0/18:1) alone (FIG. 35A), and in combination with the FOB test (FIG. 35B), for discrimination between CRC and AD.

FIG. 36. ROC curve for SM(d18:1/23:0) alone (FIG. 36A), and in combination with the FOB test (FIG. 36B), for discrimination between CRC and AD.

FIG. 37. ROC curve for SM(42:3) alone (FIG. 37A), and in combination with the FOB test (FIG. 37B), for discrimination between CRC and AD.

FIG. 38. ROC curve for TG(54:1) alone (FIG. 38A), and in combination with the FOB test (FIG. 38B), for discrimination between CRC and AD.

FIG. 39. ROC curve for the combined 7 metabolites (FIG. 39A), and in combination with the FOB test (FIG. 39B), for discrimination between CRC and AD.

FIG. 40. Extracted ion chromatogram of SM(42:3) analysed by UPLC-MS, indicating its retention time under our methodology.

FIG. 41. Extracted ion chromatogram of TG(54:1) analysed by UPLC-MS, indicating its retention time under our methodology.

FIG. 42. Extracted ion chromatogram of TG(54:3) analysed by UPLC-MS, indicating its retention time under our methodology.

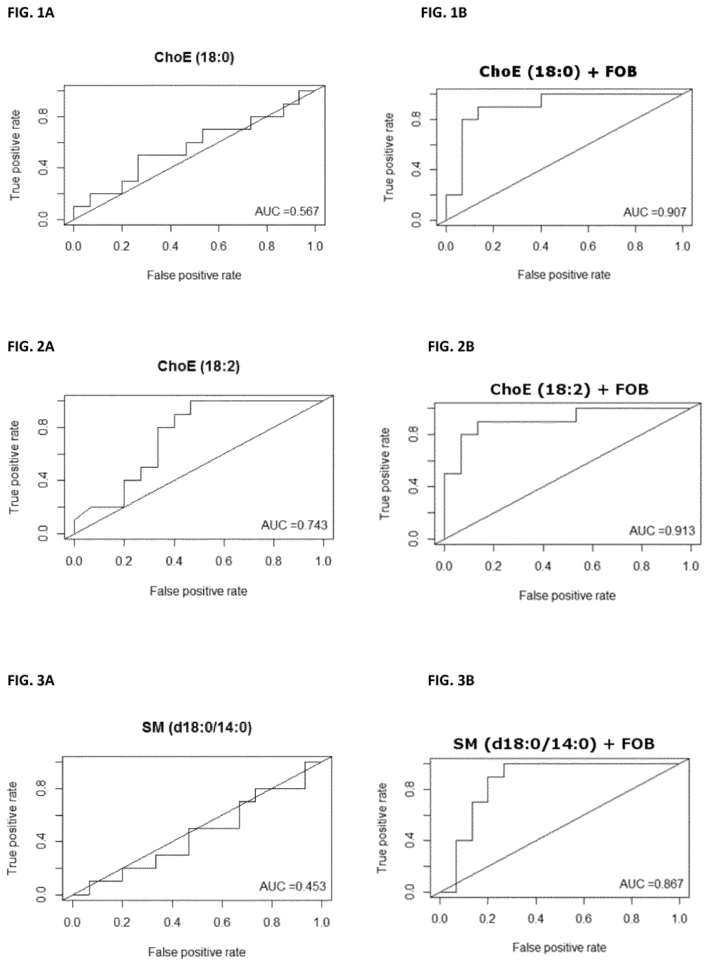

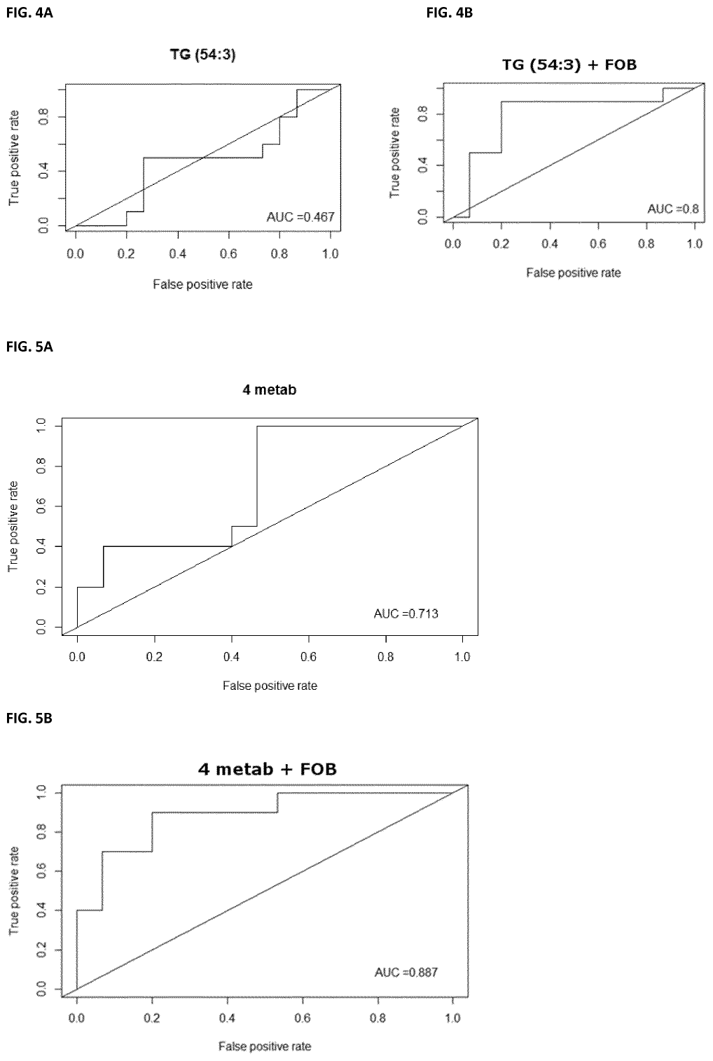

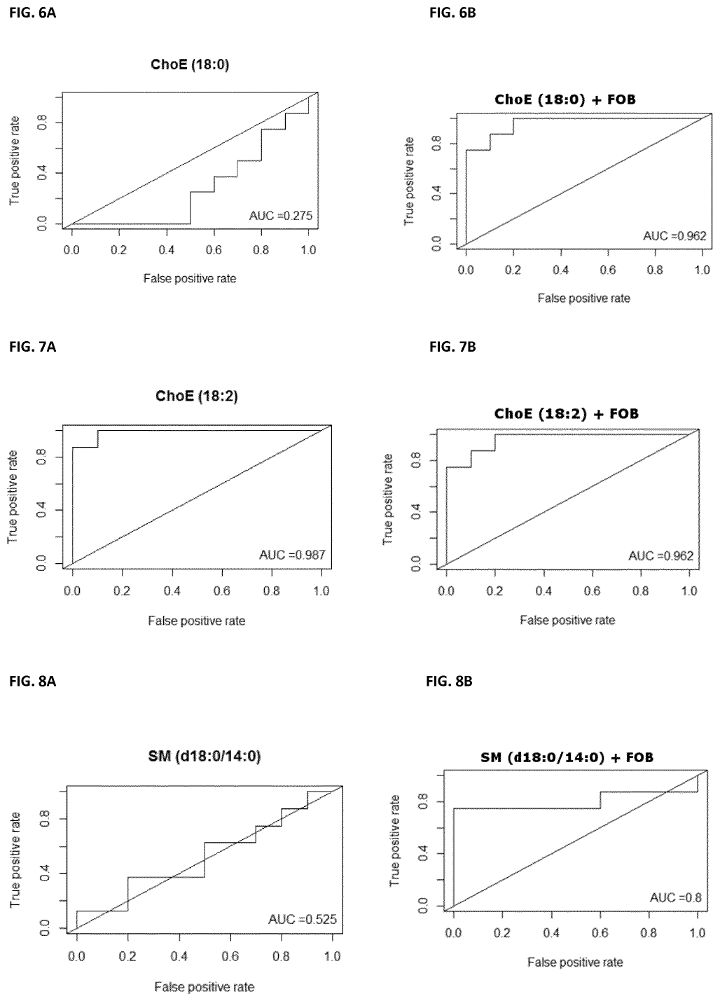

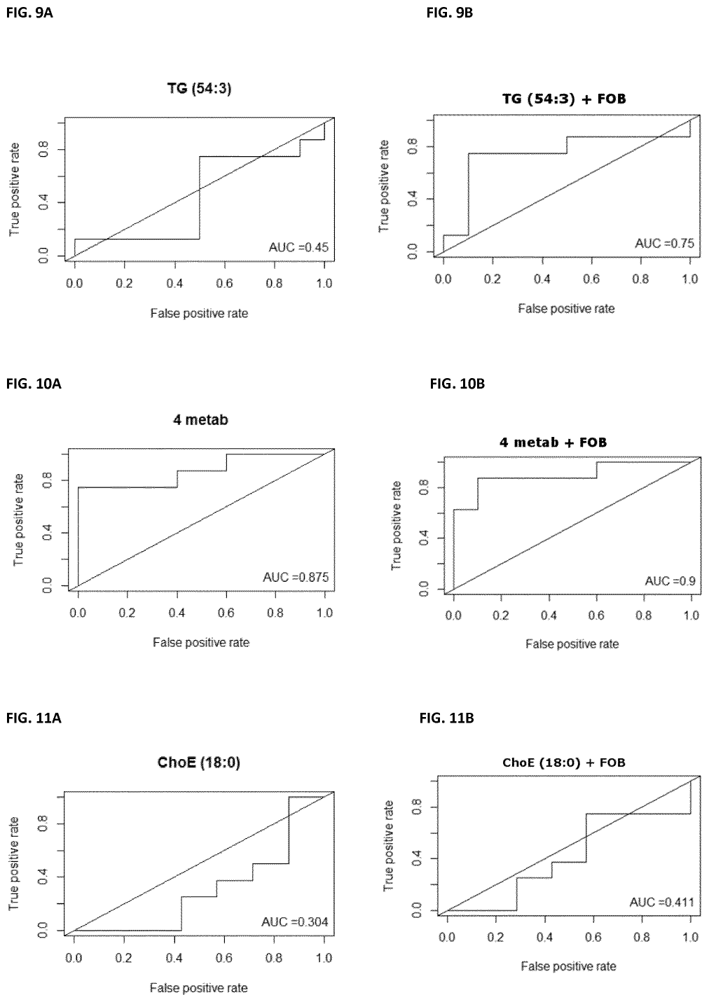

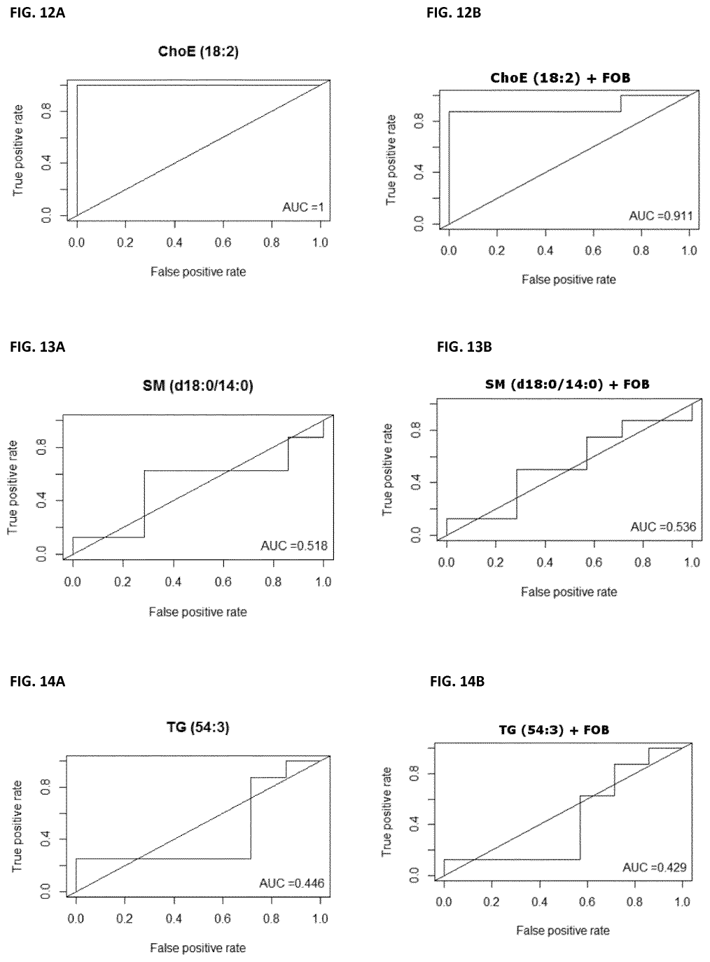

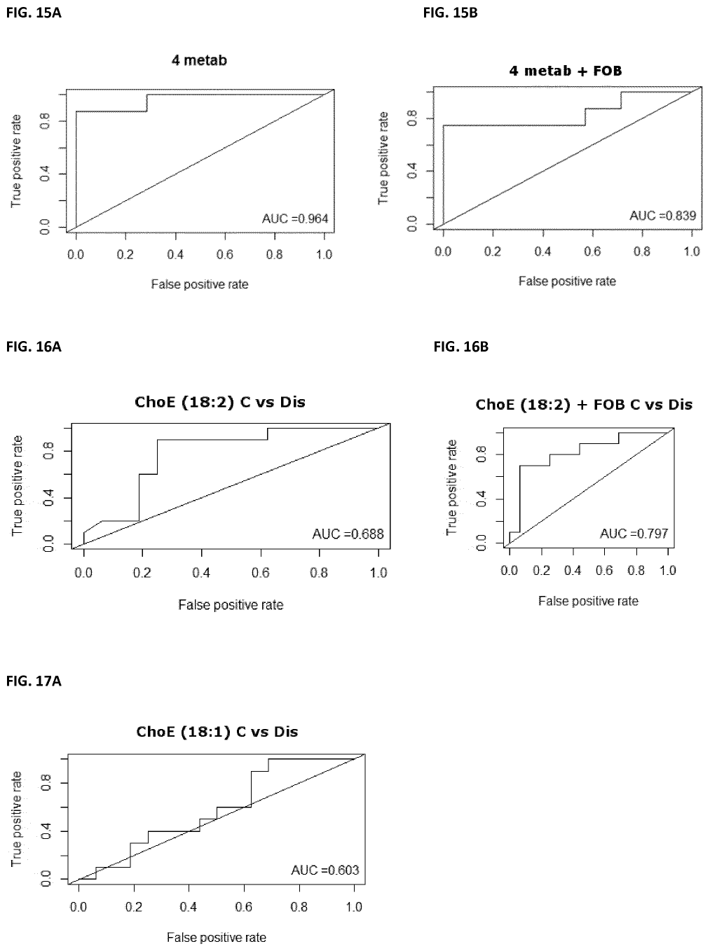

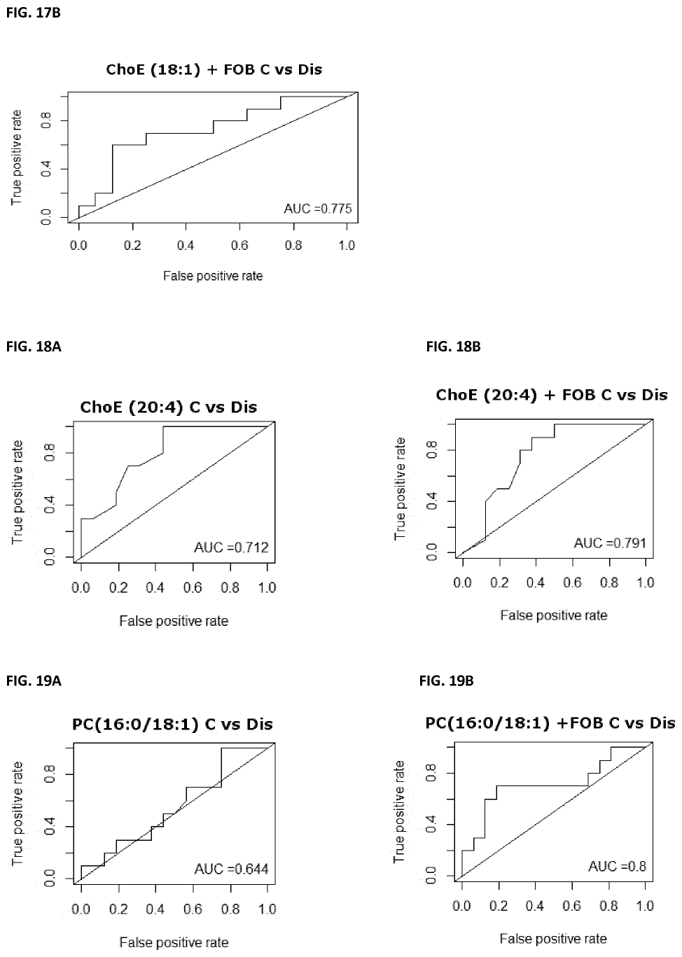

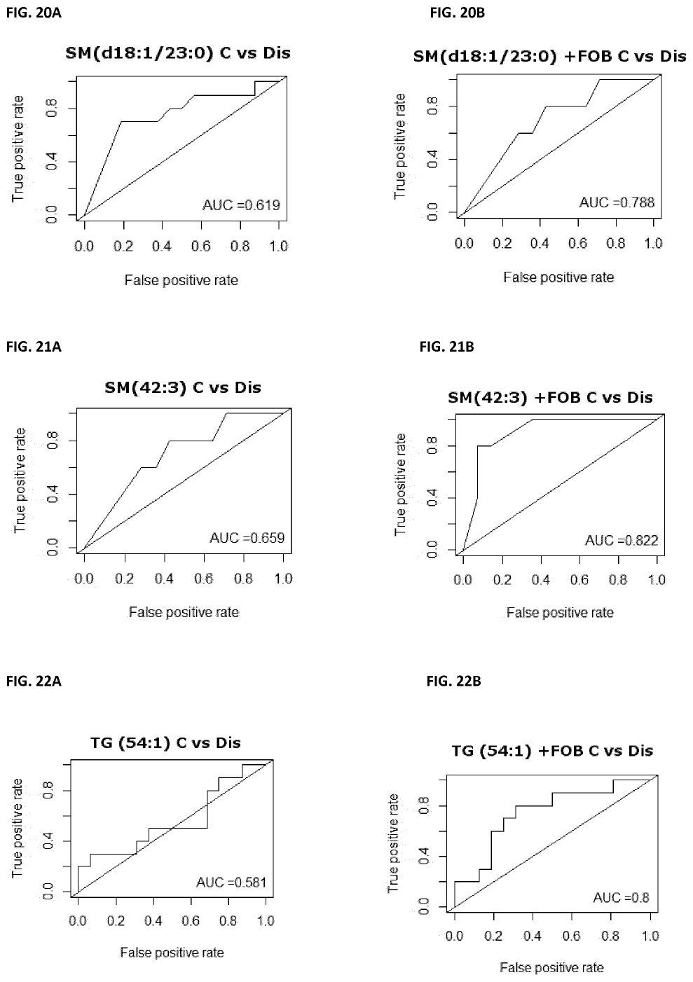

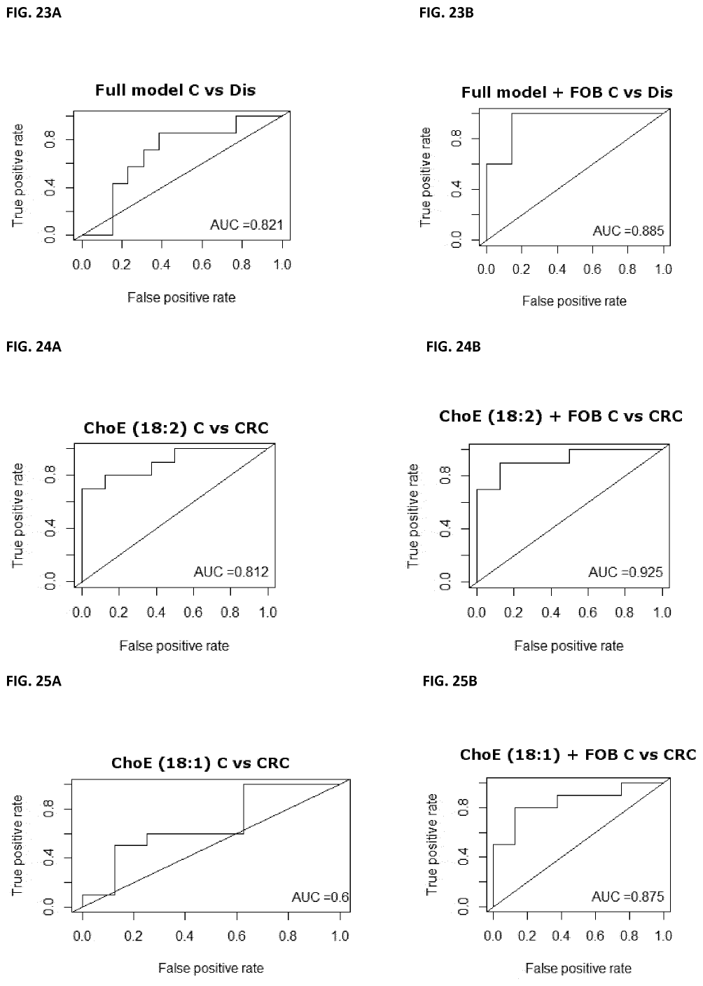

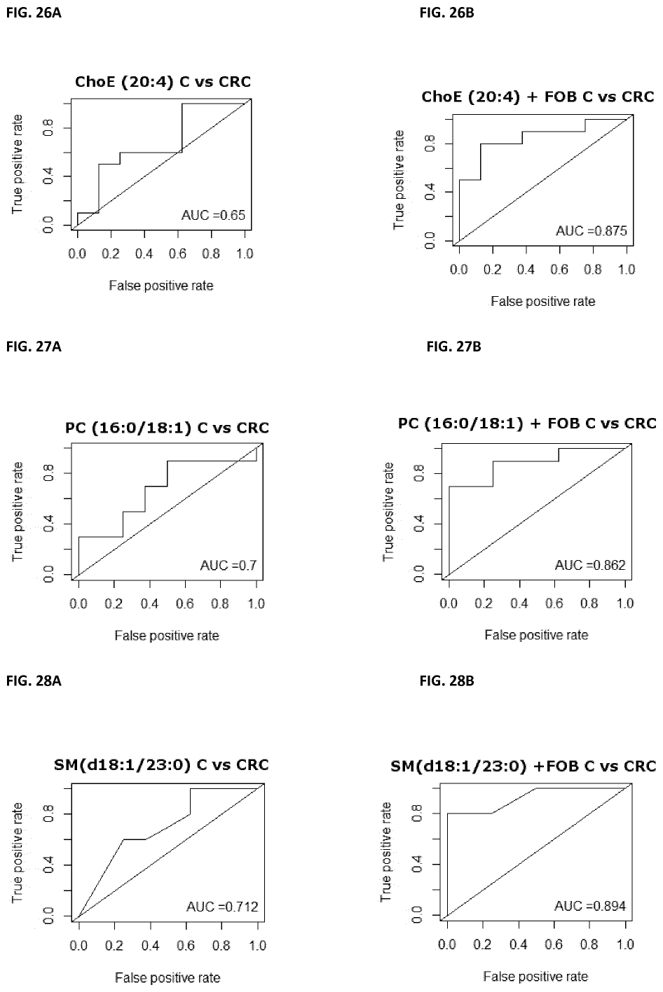

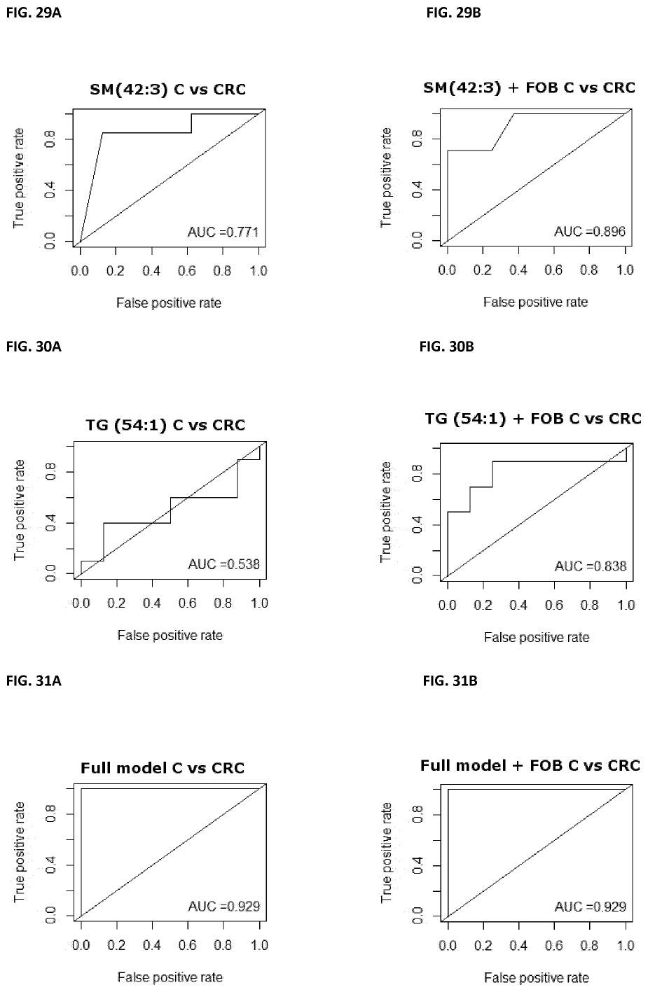

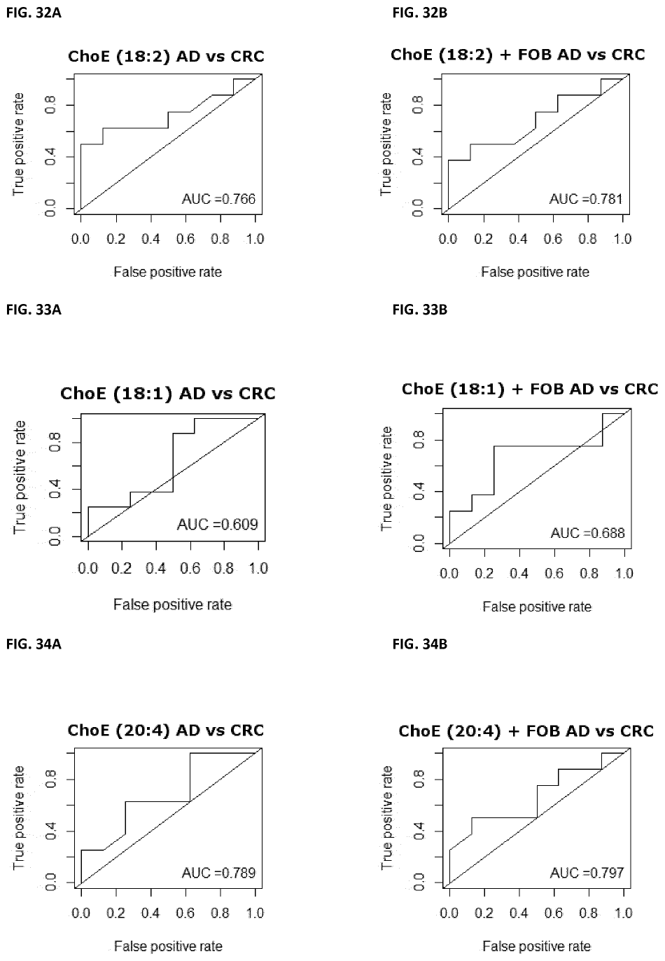

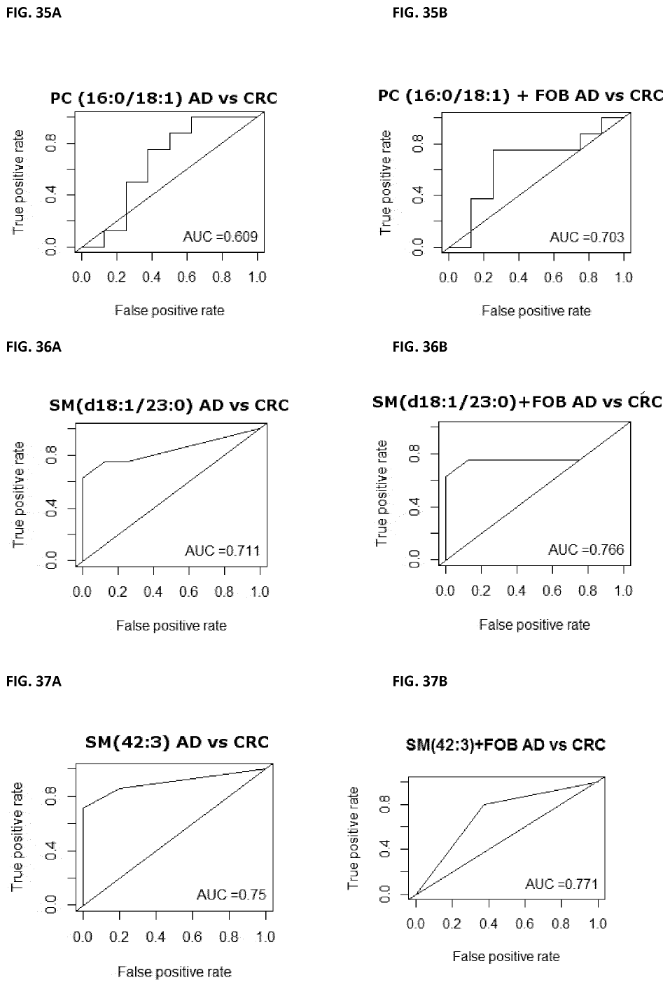

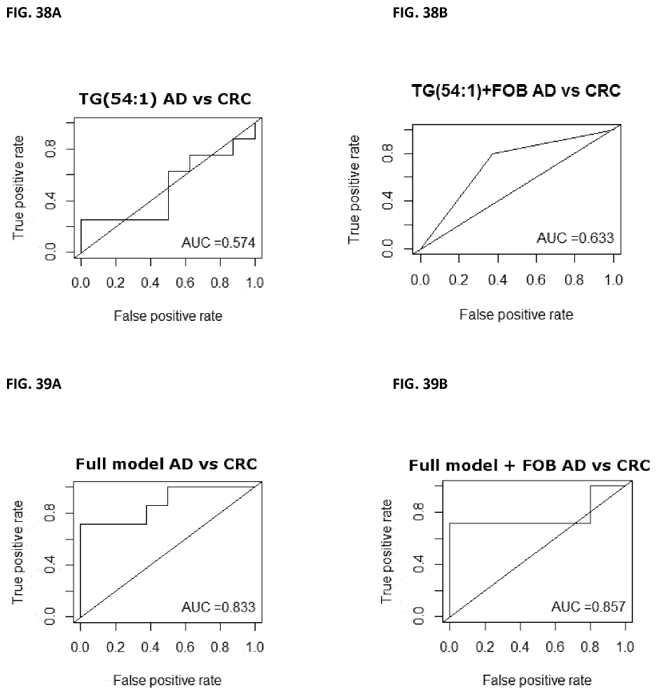

### DETAILED DESCRIPTION

Methods of the Invention

In a first aspect, the present invention provides an in vitro method for the screening, diagnosis or monitoring of colorectal cancer and/or advanced adenoma in a subject, comprising determining in a biological sample isolated from said subject the levels of at least one metabolic marker described in Table 1 and comparing the levels of said marker or markers with respect to the levels of the same marker or markers in a HC patient or with respect to the same marker or markers in an CRC or in an advanced adenoma patient or with respect to a reference value as described through-out the present specification, wherein the subject is classified as suffering CRC and/or advanced adenoma respectively, as explained throughout the specification and in the examples.

In a related aspect, it refers to an in vitro method for obtaining useful data for the screening, diagnosis or monitoring of colorectal cancer and/or advanced adenoma in a subject, said method comprising the steps defined above.

As used herein, the term “colorectal cancer”, “CRC” or “CC” is used for cancer that starts in the colon or the rectum. These cancers can also be referred to separately as colon cancer or rectal cancer, depending on where they start. Colon cancer and rectal cancer have many features in common. According to the ACS, several types of cancer can start in the colon or rectum. More than 95% of colorectal cancers are a type of cancer known as adenocarcinomas. These cancers start in cells that form glands that make mucus to lubricate the inside of the colon and rectum. Other, less common types of tumors may also start in the colon and rectum. These include: carcinoid tumors, gastrointestinal stromal tumors (GISTs), lymphomas and sarcomas. In a preferred embodiment, said colorectal cancer is adenocarcinoma.

The term “diagnosis”, as used herein, refers both to the process of attempting to determine and/or identify a possible disease in a subject, i.e. the diagnostic procedure, and to the opinion reached by this process, i.e. the diagnostic opinion. As such, it can also be regarded as an attempt at classifying an individual's condition into separate and distinct categories (such as predicting the “increasing risk” of suffering a disease, meaning “increasing risk” as an increased chance of developing or acquiring a disease compared with a normal individual) that allow medical decisions about treatment and prognosis to be made. It is to be understood that the method, in a preferred embodiment, is a method carried out in vitro, i.e. not practiced on the human or animal body. In particular, the diagnosis to determine CRC or advanced adenoma patients, relates to the capacity to identify and classify CRC or advanced adenoma patients.

In a particular embodiment, the method of diagnosis of the invention is carried out in a subject who is suspected of having CRC and/or advanced adenoma. The term “subject suspected of having CRC and/or advanced adenoma” as used herein refers to a subject that presents one or more signs or symptoms which may be indicative of CRC and/or advanced adenoma. A subject suspected of having CRC and/or advanced adenoma, further encompasses an individual who has received a preliminary diagnosis but for whom a confirmatory test (e.g., a colonoscopy) has not yet been done.

Signs or symptoms which may be indicative of CRC and/or advanced adenoma include for instance one or more of the following: unexplained weight loss, abdominal pain, unexplained rectal bleeding, iron-deficiency anemia, changes in bowel habit, and presence of occult blood in feces (see for example, Jellema et al, BMJ 2010, 340: c1269, or Adelstein et al. BMC Gastroenterology 2011, 11:65). For illustrative purposes, are provided herein below those instances where CRC is suspected in the current NICE guideline (NG12); https://www.nice.org.uk/guidance/ng12/chapter/1-Recommendations-organised-by-site-of-cancer#lower-gastrointestinal-tract-cancers:
Refer adults using a suspected cancer pathway referral (e.g., for an appointment within 2 weeks) for colorectal cancer if:
   they are aged 40 and over with unexplained weight loss and abdominal pain or
   they are aged 50 and over with unexplained rectal bleeding or
   they are aged 60 and over with:
      iron-deficiency-anemia or
      changes in their bowel habit, or
   tests show occult blood in their feces.
Consider a suspected cancer pathway referral (e.g., for an appointment within 2 weeks) for colorectal cancer in adults with a rectal or abdominal mass.
Consider a suspected cancer pathway referral (e.g., for an appointment within 2 weeks) for colorectal cancer in adults aged under 50 with rectal bleeding and any of the following unexplained symptoms or findings:
   abdominal pain
   change in bowel habit
   weight loss
   iron-deficiency anaemia.
Offer testing for occult blood in faeces to assess for colorectal cancer in adults without rectal bleeding who:
   are aged 50 and over with unexplained:
      abdominal pain or
      weight loss, or
   are aged under 60 with:
      changes in their bowel habit or
      iron-deficiency anaemia, or
   are aged 60 and over and have anaemia even in the absence of iron deficiency.

The method of the invention may also be used for the differential diagnosis between CRC and advanced adenoma.

The most effective and economic measure to reduce CRC incidence and mortality are CRC risk screening and monitoring tests. Screening tests are grouped into those that primarily detect cancer early; and those that can detect cancer early and also can detect advanced adenoma, thus providing a greater potential for prevention through polypectomy (i.e., polyps removal), see American Cancer Society (ACS) 2008 screening and surveillance guidelines, Levin et al. (CA Cancer J Clinicians, 2008; 58(3)130-160). Different screening guidelines being of application at people of average risk and at people with increased or high risk (http://www.cancer.org/cancer/colonandrectumcancer/moreinformation/colonandrectumcancerearlydetection/colorectal-cancer-early-detection-acs-recommendations).

The term “screening” is understood herein as the examination or testing of a group of asymptomatic individuals pertaining to the general population, or of a group of individuals having one or more risk factors (i.e., a subject suspected of developing or at risk of developing a disease), with the objective of discriminating healthy individuals from those who have or are suspected of having a disease. A method of screening is generally used for the “early detection” of a disease. The expression “early detection” refers to detection before the presence of clinical signs.

The goal of cancer screening is to reduce mortality through early detection and treatment thus enabling a reduction in incidence of advanced disease which generally has a worse prognosis. To this end, modern CRC screening can achieve this goal through the detection of early-stage adenocarcinomas and the detection and removal of advanced adenoma. The method of screening of the invention is thus found to be a potent tool to reduce mortality associated to CRC through early stage CRC and/or advanced adenoma detection.

Adenoma is a non-cancerous growth of abnormal glandular cells on the inner lining of an organ such as the colon. Adenomas measuring 10 mm or more in diameter, with villous architecture, high-grade dysplasia, or intramucosal carcinoma, are typically classified as advanced adenomas or advanced colorectal adenomas (Quintero E., Castells A., et al., N Engl J Med. 2012, 366(8):697-706; Cubiella J. et al., Cancer Epidemiol Biomarkers Prev. 2014, 23(9):1884-92; Muto T, Bussey H J R M B., Cancer 1975; 36:2251-70).

According to the ACS, tests that have the best chance of finding both advanced adenoma and cancer are preferred. Cohort studies involving patients with adenomas suggested that polypectomy can prevent approximately 80% of colorectal cancers (Citarda F, et al., Gut 2001, 48:812-5; Winawer S J, et al., N Engl J Med 1993, 329:1977-81).

In a particular embodiment, the method of the invention is a method of screening for the detection of CRC and advanced adenoma. In another particular embodiment, the method of the invention is a method of screening for the detection of CRC.

The method of the screening of the invention may be conducted in individuals that do not present symptoms of intestinal disease (seemingly healthy individuals). It may also be carried out in subjects with or without personal or family history of colonic neoplasm, or other risk factors of CRC as described below.

In a particular embodiment, the screening method of the invention is carried out in subjects aged 50 years and older. Average risk women and men aged 50 years and older are encouraged to follow colorectal cancer screening tests (see Table 2 of Levin et al., CA Cancer J Clinicians, 2008; 58(3)130-160, which is hereby incorporated by reference).

In another embodiment, the screening method of the invention is carried out in increased and/or high risk subjects.

According to the ACS (see Table 3 of Levin et al., CA Cancer J Clinicians, 2008; 58(3)130-160, which is hereby incorporated by reference), increased and high risk subjects may include the following:

Increased Risk
subjects with a history of polyps on prior colonoscopy;
subjects with colorectal cancer; and
subjects with a family history of colorectal cancer or adenomatous polyps.

High Risk
subjects with familial adenomatous polyposis (FAP) diagnosed by genetic testing, or suspected FAP without genetic testing;
subjects with hereditary non-polyposis colon cancer (HNPCC or Lynch syndrome), or at increased risk of HNPCC based on family history without genetic testing; and
subjects with Inflammatory bowel disease, such as chronic ulcerative colitis or Crohn's disease.

Another goal of the present invention is to provide a pre-diagnosis tool. Specifically, the present invention provides a method for screening and identifying subjects with predisposition or risk of having CRC, for instance by detecting the presence of advanced adenoma.

The term “monitoring” as used herein refers to determining the evolution of the disease and/or the efficacy of a therapy, for example determining whether there is a remission of the disease; or on the contrary whether there is disease progression or a relapse. One of the goals of the method of monitoring of the invention is to early detect relapses.

The invention further relates to a method of treating a subject suffering from CRC and/or advanced adenoma, wherein said method comprises identifying the subject to be treated by a method comprising determining in a biological sample of said subject the levels of at least one metabolic marker selected from the group consisting of ChoE(18:2), ChoE(18:0), ChoE(18:1), ChoE(20:4), PE(16:0/18:1), SM(d18:0/14:0), SM(d18:1/23:0), SM(42:3), TG(54:3) and TG(54:1), as described herein, and treating said subject suffering from CRC and/or advanced adenoma. Preferred features and embodiments are as defined throughout the specification.

CRC and/or advanced adenoma detection (in the screening, diagnosis or monitoring method of the invention), as it is understood by a person skilled in the art does not claim to be correct in 100% of the analyzed samples. However, it requires that a statistically significant amount of the analyzed samples are classified correctly. The amount that is statistically significant can be established by a person skilled in the art by means of using different statistical tools; illustrative, non-limiting examples of said statistical tools include determining confidence intervals, determining the p-value, the Chi-Square test discriminating functions, etc. Preferred confidence intervals are at least 90%, at least 95%, at least 97%, at least 98%, at least 99%. The p-values are, preferably less than 0.1, less than 0.05, less than 0.01, less than 0.005 or less than 0.0001. The teachings of the present invention preferably allow correctly diagnosing in at least 60%, in at least 70%, in at least 80%, or in at least 90% of the subjects of a determining group or population analyzed.

The terms “subject”, or “individual” are used herein interchangeably to refer to all the animals classified as mammals and includes but is not limited to domestic and farm animals, primates and humans, for example, human beings, non-human primates, cows, horses, pigs, sheep, goats, dogs, cats, or rodents. Preferably, the subject is a male or female human being of any age or race.

The term “metabolic marker”, “metabolic biomarker” or “metabolite”, are used herein interchangeably to refers to small molecule compounds, such as substrates for enzymes of metabolic pathways, intermediates of such pathways or the products obtained by a metabolic pathway, the occurrence or amount of which is characteristic for a specific situation, for example CRC or AD. The metabolic markers useful in the methods of the invention are those defined in Table 1. Table 1 contains the abbreviated common names of the metabolites and the lipid family. The lipid family is further described by the reference number of said lipid family in the LIPID MAPS structure database (http://www.lipidmaps.org/data/databases.html) using the LIPID MAPS Classification System (Fahy E. et al., Journal of Lipid Research 2009, 50: S9-S14). The lipid metabolic markers of Table 1 are intended to refer to any isomer thereof, including structural and geometric isomers. The term “structural isomer”, as used herein, refers to any of two or more chemical compounds, having the same molecular formula but different structural formulas. The term “geometric isomer” or “stereoisomer” as used herein refers to two or more compounds which contain the same number and types of atoms, and bonds (i.e., the connectivity between atoms is the same), but which have different spatial arrangements of the atoms, for example cis and trans isomers of a double bond, enantiomers, and diastereomers.

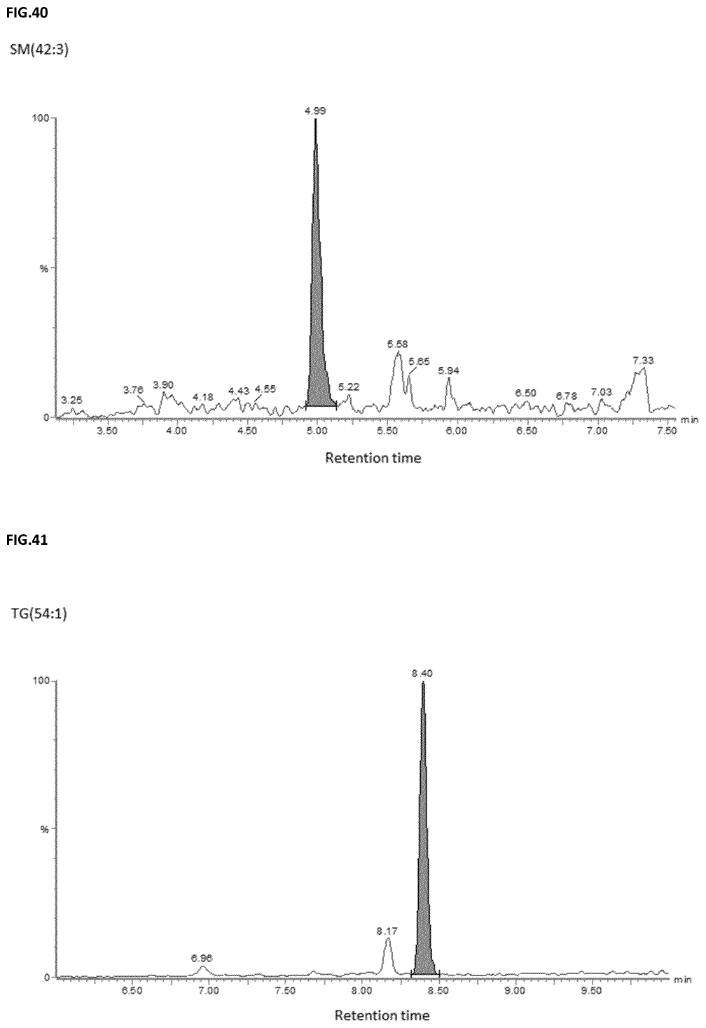

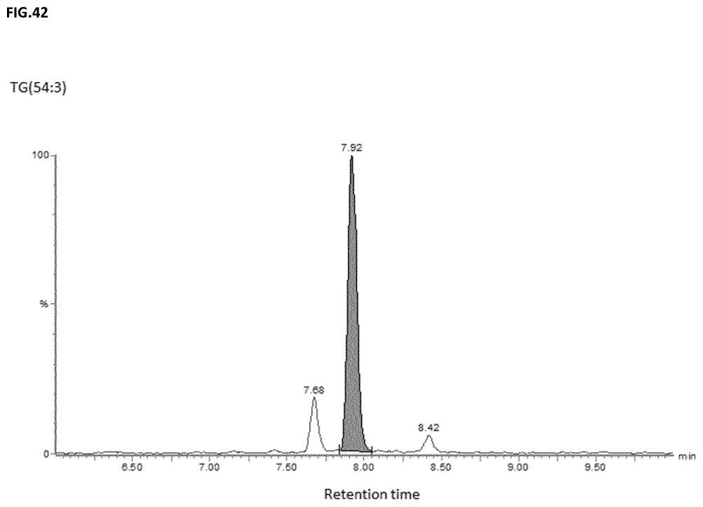

TABLE 1
Metabolites significantly differentiated between Healthy Controls and CRC and/or advanced adenoma
patients; and between CRC and advanced adenoma patients. Extracted ion chromatograms of complex
metabolites (i.e. TG(54:1), TG(54:3) and SM(42:3) are provided in FIGS. 40 to 42.

| | IUPAC name and | |
|---|---|---|
| Abbreviated or Common | Lipid Maps (LM) accession | Lipid family name and |
| name | number | Lipid Maps (LM) accession number |
| 18:0 Cholesteryl ester; | cholest-5-en-3β-yl | Steryl esters [ST0102] |
| ChoE(18:0) | octadecanoate | |
| | (LMST01020007) | |
| 18:1 Cholesteryl ester; | cholest-5-en-3β-yl (9Z- | Steryl esters [ST0102] |
| ChoE (18:1) | octadecenoate) | |
| | (LMST01020003) | |
| 18:2 Cholesteryl ester; | cholest-5-en-3β-yl (9Z, 12Z- | Steryl esters [ST0102] |
| ChoE(18:2) | octadecadienoate) | |
| | (LMST01020008) | |
| 20:4 Cholesteryl ester; | cholest-5-en-3β-yl | Steryl esters [ST0102] |
| ChoE (20:4) | (5Z, 8Z, 11Z, 14Z- | |
| | eicosatetraenoate) | |
| | (LMST01020014) | |
| Triacylglycerol 54:1; | There are two possible | Triacylglycerols [GL0301] |
| TG(54:1) | compounds with the same | |
| | mass which would fit in the | |
| | formula which cannot be | |
| | discriminated with the used | |
| | technology. TG(54:1) may | |
| | refer to any one of or a | |
| | combination of any of the | |
| | following: | |
| | TG(18:0/18:1/18:0) or | |
| | TG(18:1/18:0/18:0). | |
| Triacylglycerol 54:3; | There are various compounds | Triacylglycerols [GL0301] |
| TG(54:3) | with the same mass which | |
| | would fit in the formula which | |
| | cannot be discriminated with | |
| | the used technology. TG(54:3) | |
| | may refer to any one of or a | |
| | combination of any of the | |
| | following: TG(20:2 + 20:1 + 14:0) | |
| | or TG (20:2 + 18:1 + 16:0) or TG | |
| | (20:1 + 18:2 + 16:0) or | |
| | TG(18:2 + 18:1 + 18:0) | |
| SM(d18:0/14:0) | N-(tetradecanoyl)- | Phosphosphingolipids [SP03] |
| | sphinganine-1- | |
| | phosphocholine | |
| | (LMSP03010030) | |
| SM(d18:1/23:0) | N-(tricosanoyl)-sphing-4- | Phosphosphingolipids [SP03] |
| | enine-1- | |
| | phosphocholine | |
| | (LMSP03010078) | |
| SM(42:3) | There are 2 compounds with | phosphosphingolipids [sp03] |
| | the same mass which would fit | |
| | in the formula which cannot be | |
| | discriminated with the used | |
| | technology. SM(42:3) may | |
| | refer to any one of or a | |
| | combination of any of the | |
| | following: SM(d18:2/24:1) or | |
| | SM (d18:1/24:2) | |
| PE(16:0/18:1) | 1 -hexadecanoyl-2-(9Z- | Glycerophosphoethanolamines [GP02] |
| | octadecenoyl)-sn-glycero-3- | |
| | phosphoethanolamine | |
| | (LMGP02010009) | |

In a particular embodiment, optionally in combination with one or more of the embodiments described above or below, the method of the invention comprises determining the levels of ChoE(18:0). It is noted that a decrease of said marker with respect to HC or with respect to a reference value is indicative that the subject suffers from CRC; and an increase of said marker with respect to HC or with respect to a reference value is indicative that the subject suffers from advanced adenoma.

In another particular embodiment, optionally in combination with one or more of the embodiments described above or below, the method of the invention comprises determining the levels of ChoE (18:1). It is noted that an increase of said marker with respect to HC or with respect to a reference value is indicative that the subject suffers from CRC and/or advanced adenoma.

In a further particular embodiment, optionally in combination with one or more of the embodiments described above or below, the method of the invention comprises determining the levels of ChoE(18:2). It is noted that an increase of said marker with respect to HC or with respect to a reference value is indicative that the subject suffers from CRC and/or advanced adenoma, preferably from CRC.

In another further particular embodiment, optionally in combination with one or more of the embodiments described above or below, the method of the invention comprises determining the levels of ChoE (20:4). It is noted that an increase of said marker with respect to HC or with respect to a reference value is indicative that the subject suffers from CRC and/or advanced adenoma.

In an additional particular embodiment, optionally in combination with one or more of the embodiments described above or below, the method of the invention comprises determining the levels of TG(54:1). It is noted that a decrease of said marker with respect to HC or with respect to a reference value is indicative that the subject suffers from CRC and/or advanced adenoma.

In also a particular embodiment, optionally in combination with one or more of the embodiments described above or below, the method of the invention comprises determining the levels of TG(54:3). It is noted that a decrease of said marker with respect to HC or with respect to a reference value is indicative that the subject suffers from CRC and/or advanced adenoma, preferably from advanced adenoma.

In still a further particular embodiment, optionally in combination with one or more of the embodiments described above or below, the method of the invention comprises determining the levels of SM(d18:0/14:0). It is noted that an increase of said marker with respect to HC or with respect to a reference value is indicative that the subject suffers from CRC and/or advanced adenoma.

In another particular embodiment, optionally in combination with one or more of the embodiments described above or below, the method of the invention comprises determining the levels of SM(d18:1/23:0). It is noted that an increase of said marker with respect to HC or with respect to a reference value is indicative that the subject suffers from CRC and/advanced adenoma.

In an additional particular embodiment, optionally in combination with one or more of the embodiments described above or below, the method of the invention comprises determining the levels of SM(42:3). It is noted that an increase of said marker with respect to HC or with respect to a reference value is indicative that the subject suffers from CRC and/advanced adenoma.

It is thus herein disclosed a method which comprises determining the levels of any possible combination of two or more, including all, of ChoE(18:0), ChoE (18:1), ChoE(18:2), ChoE (20:4), TG(54:1), TG(54:3), SM(d18:0/14:0), SM(d18:1/23:0) and SM(42:3).

The term “sample” or “biological sample”, as used herein, refers to biological material isolated from a subject. The biological sample may contain any biological material suitable for detecting the desired biomarker and may comprise cellular and/or non-cellular material from the subject. The sample can be isolated from any suitable biological tissue or fluid such as, for example, blood, blood plasma, serum, cerebral spinal fluid (CSF), urine, amniotic fluid, lymph fluids, external secretions of the respiratory, intestinal, genitourinary tracts, tears, saliva, white blood cells. Preferably, the samples used for the determination of the level(s) of the metabolic markers in the methods of the invention are samples which can be obtained using minimally invasive procedures. In a preferred embodiment, the samples are intestinal samples. These may be for instance a biopsy from the mucosal tissue of the colon and/or rectum. Preferably, this intestinal sample is a feces sample.

These types of samples are routinely used in the clinical practice and a person skilled in the art will know how to identify the most appropriate means for their obtaining and preservation. Once a sample has been obtained, it may be used fresh, it may be frozen, lyophilized or preserved using appropriate means. In a particular embodiment, of the methods of the invention said sample is a lyophilized feces sample. Preferably, chloroform/methanol extracts are obtained for the metabolomics analysis.

The expression “determining the levels of the marker”, as used herein, refers to ascertaining the absolute or relative amount or concentration of the biomarker in the sample. Techniques to assay levels of individual biomarkers from test samples are well known to the skilled technician, and the invention is not limited by the means by which the components are assessed. Suitable methods for determining the levels of a given metabolite include, without limitation, refractive index spectroscopy (RI), Ultra-Violet spectroscopy (UV), fluorescent analysis, radiochemical analysis, Infrared spectroscopy (IR), Nuclear Magnetic Resonance spectroscopy (NMR), Light Scattering analysis (LS), Mass Spectrometry (MS) and MS-based methods, Pyrolysis Mass Spectrometry, Nephelometry, and Dispersive Raman Spectroscopy.

The term “mass spectrometry (MS)-based methods” as used herein refers to mass spectrometry alone or coupled to other detection or separation methods, including gas chromatography combined with mass spectroscopy, liquid chromatography combined with mass spectroscopy, supercritical fluid chromatography combined with mass spectroscopy, ultra-performance liquid chromatography combined with mass spectrometry, MALDI combined with mass spectroscopy, ion spray spectroscopy combined with mass spectroscopy, capillary electrophoresis combined with mass spectrometry, NMR combined with mass spectrometry and IR combined with mass spectrometry. These MS-based methods may include single MS or tandem MS.

Mass spectrometers operate by converting the analyte molecules to a charged (ionized) state, with subsequent analysis of the ions and any fragment ions that are produced during the ionization process, on the basis of their mass to charge ratio (m/z). Several different technologies are available for both ionization and ion analysis, resulting in many different types of mass spectrometers with different combinations of these two processes. On the one hand, examples of ion sources include electrospray ionization source, atmospheric pressure chemical ionization source and atmospheric pressure photo-ionization. On the other hand, mass spectrometers analyzers may be, but are not limited to, quadrupole analyzers, time-of-flight (TOF) analyzers, ion trap analyzers or hybrid analyzers, such as hybrid quadrupole time-of-flight (QTOF) analyzers or hybrid triple quadrupole linear ion trap analyzers. In a particular embodiment, the levels of the metabolic markers are determined by ultra-performance liquid chromatography/time-of-flight mass spectrometry (UPLC-TOFMS). Preferred UPLC-TOFMS detection conditions are as described by Barr et al. (J Proteome Res, 2012, 11, 2521-2532).

Internal standards may be used in the MS analysis as it enables to correct for any losses or inefficiencies in the sample preparation process and reduce the effect of ion suppression. Stable isotope versions of the analyte are ideal internal standards as they have almost identical chemical properties but are easily distinguished during MS. In a preferred embodiment, optionally in combination with one or more of the embodiments described above or below, the determination of the levels of the metabolic marker is conducted by an MS-based method using isotope versions of the metabolic marker as internal standards.

In addition, or alternatively, the extraction solvent may be spiked with compounds not detected in unspiked human stool samples, for instance SM(d18:1/16:0), PE(17:0/17:0), PC(19:0/19:0), TAG(13:0/13:0/13:0), Cer(d18:1/17:0) and ChoE(12:0).

The diagnostic method of the invention comprises comparing the level(s) of the metabolic marker(s) with a reference value.

The term “reference value”, as used herein, relates to a predetermined criteria used as a reference for evaluating the values or data obtained from the samples collected from a subject. The reference value or reference level can be an absolute value, a relative value, a value that has an upper or a lower limit, a range of values, an average value, a median value, a mean value, or a value as compared to a particular control or baseline value. A reference value can be based on an individual sample value or can be based on a large number of samples, such as from population of subjects of the chronological age matched group, or based on a pool of samples including or excluding the sample to be tested.

The reference value according to the method of the invention can be obtained from one or more subjects not having CRC or advanced adenoma, preferably not having a gastrointestinal disease (i.e., healthy control subjects), from subjects suffering from advanced adenoma, from subjects suffering from CRC at early stage, such as non-symptomatic (preclinical stage) or from the same subject that was diagnosed as having CRC or advanced adenoma but at an earlier time point.

In a particular embodiment, the method of the invention is a method of screening or diagnosis of CRC and/or advanced adenoma and the reference value is obtained from subjects not having CRC or advanced adenoma, preferably not having a gastrointestinal disease.

In another particular embodiment, the method of the invention is a method of screening or diagnosis of CRC and the reference value is obtained from subjects not suffering from CRC or advanced adenoma.

In a further particular embodiment, the method of the invention is a method of screening or diagnosis of CRC and the reference value is obtained from subjects suffering from advanced adenoma. This may be the case for instance when differential diagnosis between CRC and advanced adenoma is desired.

In an additional particular embodiment, the method of the invention is a method of monitoring CRC and/or advanced adenoma and the reference value is obtained from the same subject that was diagnosed as having CRC or advanced adenoma but at an earlier time point.

In the methods of the invention, the level of a metabolic marker is considered “decreased” when the level of said marker in a sample is lower than its reference value. The level of a marker is considered to be lower than its reference value when it is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, or more lower than its reference value.

Likewise, in the context of the methods of the invention, the level of a marker is considered “increased” when the level of said marker in a sample is higher than its reference value. The level of a marker is considered to be higher than its reference value when it is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, or more higher than its reference value.

Alternatively or in addition, subjects having more than about 1.2, 1.3, 1.4, 1.5, 2, 3, 4, 5, 6, 7, 8, 9, 10, 15 or 20 fold levels deviation (i.e., increase or decrease) than the reference value as described herein (e.g., of an appropriate unaffected population) may be identified as having CRC and/or advanced adenoma.

In a preferred embodiment, the present invention provides an in vitro method for the screening, diagnosis or monitoring of colorectal cancer and/or advanced adenoma in a subject, said method comprising the following steps:
a) determining the levels of the metabolic marker ChoE(18:2) in a biological sample isolated from said subject;
b) comparing the levels in said biological sample with a reference value;

wherein an increase of ChoE(18:2) levels in the subject sample with regard to said reference value is indicative of colorectal cancer and/or advanced adenoma.

Said method may further comprise the determination of the levels of at least one further metabolic marker, preferably all, selected from the group consisting of ChoE(18:0), TG(54:3), SM(d18:0/14:0), ChoE(18:1), ChoE(20:4), PE(16:0/18:1), SM(d18:1/23:0), SM(42:3) and TG(54:1); and comparing the levels in said feces sample for said metabolic marker with a reference value.

In a preferred embodiment, said further metabolic marker is selected from the group consisting of ChoE(18:0), SM(d18:0/14:0) and TG(54:3). The combined use of ChoE(18:2), ChoE(18:0), SM(d18:0/14:0) and TG(54:3) enables to discriminate between HC and disease (CRC+AD) with an AUC of 0.713, between HC and CRC with an AUC of 0.875 and between CRC and AD with an AUC of 0.964 (see Table 4). In a more preferred embodiment, the method of the invention comprises the quantification of ChoE(18:2), ChoE(18:0), SM(d18:0/14:0) and TG(54:3).

In another preferred embodiment, said further metabolic marker is selected from the group consisting of ChoE(18:1), ChoE(20:4), PE(16:0/18:1), SM(d18:1/23:0), SM(42:3) and TG(54:1).

The combined use of ChoE(18:2), ChoE(18:1), ChoE(20:4), PE(16:0/18:1), SM(d18:1/23:0), SM(42:3) and TG(54:1)enables to discriminate between HC and disease (CRC+AD) with an AUC of 0.821, between HC and CRC with an AUC of 0.929 and between CRC and AD with an AUC of 0.833 (see Table 6). In a more preferred embodiment, the method of the invention comprises the quantification of ChoE(18:2), ChoE(18:1), ChoE(20:4), PE(16:0/18:1), SM(d18:1/23:0), SM(42:3) and TG(54:1).

It is further noted that the accuracy of the method of the invention can be increased by determining the presence and/or quantification of other biomarkers.

The term “biomarker” as used herein refers to markers of disease which are typically substances found in a bodily sample that can be easily measured. Said bodily sample can be for instance a blood, plasma or feces sample. Typically, the measured amount correlates to an underlying disease pathophysiology, such as presence or absence of CRC and/or advanced adenoma, making it useful for diagnosing and measuring the progress of a disease or the effects of a treatment. The term biomarker encompasses biophysical and biochemical determinations, including genetic and serological markers.

Preferably, said other biomarker is determined in a feces sample. For instance, fecal occult blood (FOB) tests, such as the guaiac-based fecal occult blood test (gFOBT) and the fecal immunochemical test (“FIT”), or the stool DNA (“sDNA”) test (see, Quintero E., Castells A., et al., N Engl J Med. 2012, 366(8):697-706; Cubiella J. et al., Cancer Epidemiol Biomarkers Prev. 2014, 23(9):1884-92; Levin B et al., Gastroenterology 2008; 134:1570-95).

In a particular embodiment, optionally in combination with one or more embodiments as described above or below, the method of the invention further comprises conducting a fecal occult blood (FOB) test. In a preferred embodiment, said FOB test is a fecal immunochemical test (“FIT”) which detects human globin. A variety of FIT exist based on different detection technologies (e.g., reversed passive haemagglutination (RPHA), latex agglutination, ELISA and immunonephelometry) and are encompassed herein (Levin B et al., Gastroenterology 2008; 134:1570-95). Preferably, said immunologic test is based on a latex agglutination reaction, such as the OC-SENSOR MICRO test used in the Examples.

The AUC, sensitivity and specificity results for each of the metabolic markers selected from the group consisting of ChoE(18:2), ChoE(18:0), TG(54:3), SM(d18:0/14:0), ChoE(18:1), ChoE(20:4), PE(16:0/18:1), SM(d18:1/23:0), SM(42:3) and TG(54:1) in combination with the FOB test (for each of the relevant comparisons, namely HC vs disease (CRC+AD), HC vs CRC and CRC vs AD) are provided in Tables 4 and 7.

It has been found by the inventors that the combined use of ChoE(18:2), ChoE(18:0), SM(d18:0/14:0) and TG(54:3) with the FOB test enables to discriminate between HC and disease (CRC+AD) with an AUC of 0.887, between HC and CRC with an AUC of 0.800 and between CRC and AD with an AUC of 0.839.

It has also been found by the inventors that the combined use of ChoE(18:2), ChoE(18:1), ChoE(20:4), PE(16:0/18:1), SM(d18:1/23:0), SM(42:3) and TG(54:1) with the FOB test enables to discriminate between HC and disease (CRC+AD) with an AUC of 0.885, between HC and CRC with an AUC of 0.929 and between CRC and AD with an AUC of 0.857.

Exploratory tests such as colonoscopy, flexible sigmoidoscopy, double-contrast barium enema and/or computed tomography (CT) colonography may further be conducted for confirmatory purposes.

The methods of the present invention might be implemented by a computer. Therefore, a further aspect of the invention refers to a computer implemented method, wherein the method is any of the methods disclosed herein or any combination thereof.

It is noted that any computer program capable of implementing any of the methods of the present invention or used to implement any of these methods or any combination thereof, also forms part of the present invention.

It is also noted that any device or apparatus comprising means for carrying out the steps of any of the methods of the present invention or any combination thereof, or carrying a computer program capable of, or for implementing any of the methods of the present invention or any combination thereof, is included as forming part of the present specification.

The methods of the invention may also comprise the storing of the method results in a data carrier, preferably wherein said data carrier is a computer readable medium. The present invention further relates to a computer-readable storage medium having stored thereon a computer program of the invention or the results of any of the methods of the invention.

As used herein, “a computer readable medium” can be any apparatus that may include, store, communicate, propagate, or transport the results of the determination of the method of the invention. The medium can be an electronic, magnetic, optical, electromagnetic, infrared, or semiconductor system (or apparatus or device) or a propagation medium.

Kit of the Invention

A second aspect, the present invention refers to a kit for determining the levels of one or more of the metabolic markers as described in Table 1 in a biological sample (preferably a feces sample) isolated from a subject. The kit may also contain instructions indicating how the materials within the kit may be used.

The term “kit” or “testing kit” denotes combinations of reagents and adjuvants required for an analysis. Although a test kit consists in most cases of several units, one-piece analysis elements are also available, which must likewise be regarded as testing kits.

In a particular embodiment of the second aspect, said kit is suitable for determining the levels of at least ChoE(18:2) in a biological sample and comprises:
a) a reagent for determining the levels of ChoE(18:2), and optionally a reagent for determining the levels of at least one further metabolic marker, preferably all, selected from the list consisting of ChoE(18:0), TG(54:3), SM(d18:0/14:0), ChoE(18:1), ChoE(20:4), PE(16:0/18:1), SM(d18:1/23:0), SM(42:3) and TG(54:1), in a biological sample;
b) optionally, instructions for the use of said reagent(s) in determining the levels of ChoE(18:2), and optionally of at least said one further metabolic marker, in a biological sample.

Preferably, the further metabolic marker of step a) is selected from the group consisting of ChoE(18:0), SM(d18:0/14:0), ChoE(18:1), ChoE(20:4), PE(16:0/18:1), and SM(d18:1/23:0).

In a preferred embodiment, said further metabolic marker is selected from the group consisting of ChoE(18:0), SM(d18:0/14:0) and TG(54:3), preferably from the group consisting of ChoE(18:0), and SM(d18:0/14:0). In another preferred embodiment, said further metabolic marker is selected from the group consisting of ChoE(18:1), ChoE(20:4), PE(16:0/18:1), SM(d18:1/23:0), SM(42:3) and TG(54:1), preferably from the group consisting of ChoE(18:1), ChoE(20:4), PE(16:0/18:1), and SM(d18:1/23:0).

In a particular embodiment, optionally in combination with one or more of the embodiments described above or below, the determination of the levels of said metabolic marker is carried out by a mass spectrometry (MS)-based method, and said kit comprises said metabolic marker unlabeled and/or said metabolic marker stably labelled for detection by a mass spectrometry (MS)-based method, preferably wherein the metabolic marker is labelled with a tag which comprises one or more stable isotope. Isotopic atoms which may be incorporated into the tag are heavy atoms for example ¹³C, ¹⁵N, ¹⁷O and/or ³⁴S, which can be distinguished by MS. The unlabeled markers, although cannot be spiked into the sample, may be used for the qualitative identification and absolute quantitation (by performing a calibration curve with increasing amounts of the marker) of the peaks corresponding to each of the markers and/or to facilitate the establishment of the chromatographic and mass spectrometric conditions optimal for detection of the markers. On the other hand, the stably labelled marker can be spiked into the sample and used as internal standard to obtain chromatographic features and the absolute concentration of the markers of interest.

Preferably, said kit is suitable for determining the levels of at least ChoE(18:2) and the kit comprises a labelled and/or unlabeled ChoE(18:2) for detection by a mass spectrometry (MS)-based method, preferably a isotope labelled ChoE(18:2).

In a preferred embodiment, optionally in combination with one or more of the embodiments described above or below, said kit is suitable for determining the levels of at least ChoE(18:2) in a feces sample, and comprises:
a) a labelled and/or unlabeled ChoE(18:2), and optionally at least one labelled and/or unlabeled further metabolic marker, preferably all, selected from the list consisting of ChoE(18:0), TG(54:3), SM(d18:0/14:0), ChoE(18:1), ChoE(20:4), PE(16:0/18:1), SM(d18:1/23:0), SM(42:3) and TG(54:1), in a feces sample;
b) optionally, instructions for the use of said reagent in determining the levels of ChoE(18:2), and optionally of at least said one further metabolic marker, in a feces sample.

In a further preferred embodiment, optionally in combination with one or more of the embodiments described above or below, said kit is suitable for determining the levels of at least ChoE(18:2) in a feces sample, and comprises:
a) a isotope labelled ChoE(18:2), and optionally isotope labelled versions of at least one further metabolic marker, preferably all, selected from the list consisting of ChoE(18:0), TG(54:3), SM(d18:0/14:0), ChoE(18:1), ChoE(20:4), PE(16:0/18:1), SM(d18:1/23:0), SM(42:3) and TG(54:1), in a feces sample;
b) optionally, instructions for the use of said reagent in determining the levels of ChoE(18:2), and optionally of at least said one further metabolic marker, in a feces sample.

The further metabolic marker of step a) may be as described throughout the specification. Preferably, it is selected from the group consisting of ChoE(18:0), SM(d18:0/14:0), ChoE(18:1), ChoE(20:4), PE(16:0/18:1), and SM(d18:1/23:0).

Optionally, the kit can also include appropriate tubes and solvents for feces extraction, e.g., a chloroform/methanol solution and/or solvents for chromatographic-MS analysis, e.g., an acetronitrile/isopropanol solution.

Other preferred features and embodiments of the kit of the invention are as described herein throughout the specification.

A third aspect of the invention refers to the in vitro use of the kit as defined in the second aspect of the invention or in any of its preferred embodiments, for the screening, diagnosis or monitoring of colorectal cancer and/or advanced adenomas in a subject.

A fourth aspect of the invention refers to a computer program product directly loadable into the internal memory of a digital computer, comprising software code portions for performing the steps of comparing the level of one or more of the metabolic markers of Table 1, as described under the first aspect of the invention, from the one or more biological samples of a subject with a reference value and determining whether the subject is suffering from CRC and/or advanced adenomas, when said product is run on a computer.

A fifth aspect of the invention relates to a method of measuring the levels of a metabolic marker as described in Table 1 in a biological sample (preferably a feces sample) of a subject, said method comprising:
a) obtaining at least a biological sample from a subject; and
b) measuring the levels of at least one of said metabolic markers in said biological sample by a mass spectrometry-based method.

A particular embodiment concerns a method of measuring the levels of ChoE(18:2) in a feces sample of a subject, said method comprising:
a) obtaining at least a feces sample from a subject; and
b) measuring the levels of ChoE(18:2) in said feces sample by a mass spectrometry-based method.

In a preferred embodiment, step b) further comprises measuring the levels of at least one further metabolic marker, preferably all, selected from the list consisting of ChoE(18:0), TG(54:3), SM(d18:0/14:0), ChoE(18:1), ChoE(20:4), PE(16:0/18:1), SM(d18:1/23:0), SM(42:3) and TG(54:1) by a mass spectrometry-based method.

Other preferred features and embodiments of the method of measuring the levels of a metabolic marker of the invention are as described herein for other aspects of the invention.

It is contemplated that any features described herein can optionally be combined with any of the embodiments of any method, kit, use of a kit, or computer program of the invention; and any embodiment discussed in this specification can be implemented with respect to any of these. It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications are herein incorporated by reference to the same extent as if each individual publication or patent application was specifically and individually indicated to be incorporated by reference.

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “comprises” also encompasses and expressly discloses the terms “consists of” and “consists essentially of”. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by ±1, 2, 3, 4, 5, 6, 7, 8, 9, 10, 11, 12, 13, 14 or 15%. Accordingly, the term “about” may mean the indicated value ±5% of its value, preferably the indicated value ±2% of its value, most preferably the term “about” means exactly the indicated value (±0%).

The following examples serve to illustrate the present invention and should not be construed as limiting the scope thereof.

### EXAMPLES

Material and Methods

The biomarker assessment in this study was organized in sequential and consecutive phases for discovery and biological validation. Firstly, 133 metabolites including glycerolipids, glycerophospholipids, sterol lipids and sphingolipids were selected as candidate biomarkers for the initial analysis of feces samples from advanced neoplasia cases and cancer-free controls (Discovery Phase). Secondly, the potential clinical use of the most promising validated candidates was tested in faeces samples from colon cancer cases, a small set of adenomas, and cancer-free controls. Reported STARD guidelines were the basis for defining the protocol. The results of the second phase are reported herein.

Chemicals

HPLC-MS grade solvents were purchase from Sigma Aldrich (St. Louis, Mo.). Reference metabolite standard compounds were obtained from Sigma Aldrich, Larodan Fine Chemicals (Malmo, Sweden) and Avanti Polar Lipids (Alabaster, Ala.).

Clinical Samples and Study Population

Feces samples from 40 patients with advanced adenomas (AD), 40 with colorectal cancer (CRC) and 49 age-matched healthy individuals were collected and lyophilized. On these samples, metabolomics profiling using UPLC-MS approach was performed. Study participants transported samples to the laboratory within 24 hours after collection which were stored at −80° C. Stools were lyophilized before proceeding with the metabolomics analysis.

Table 2 provides the main clinical features (sex, age, tumour stage, size, location) of the patients involved in the study. Tumour staging was performed according to the American Joint Committee on Cancer (AJCC) classification (Edge A, e al., AJCC Cancer Staging Manual, 7th edition. Berlin: Springer, 2010). Adenomas measuring 10 mm or more in size with villous architecture and/or high-grade dysplasia were classified as advanced adenomas. Advanced neoplasm (AN) was defined as an advanced adenoma or cancer.

TABLE 2

| | Gender | | Colorectal | Significant | | Advanced | Tumour, Node, Metastasis (TNM, |
|---|---|---|---|---|---|---|---|
| | (0 = | | cancer | colon lession | Significant | CRC_Localization | international classification), |
| | male, | | (CRC), | (LCS), | adenoma. | (1 = rectal; | 1 = 0 (intramucosal carcinoma), |
| | 1 = | | 0 = No, | 0 = No, | 0 = No, | 2 = distal; | 2 = Stage I , 3 = stage II, 4 = |
| Sample ID | female) | Age | 1 = Yes | 1 = Yes | 1 = Yes | 3 = proximal) | stage III, 5 = stage IV) |
| 13000398 | 1 | 70 | 1 | 0 | 0 | 1 | 3 |
| 13000494 | 1 | 66 | 1 | 0 | 0 | 3 | 5 |
| 13000097 | 1 | 70 | 1 | 0 | 0 | 1 | 1 |
| 13000139 | 1 | 82 | 1 | 0 | 0 | 1 | 4 |
| 13000140 | 1 | 72 | 1 | 0 | 0 | 2 | 5 |
| 13000142 | 1 | 79 | 1 | 0 | 0 | 1 | 5 |
| 13000150 | 1 | 51 | 1 | 0 | 0 | 1 | 3 |
| 13000164 | 1 | 77 | 1 | 0 | 0 | 3 | 3 |
| 13000467 | 1 | 72 | 1 | 1 | 1 | 1 | 4 |
| 13000512 | 1 | 86 | 1 | 0 | 0 | 2 | 2 |
| 13000516 | 1 | 83 | 1 | 0 | 0 | 1 | 4 |
| 13000520 | 1 | 83 | 1 | 0 | 0 | 3 | 3 |
| 13000555 | 1 | 79 | 1 | 0 | 0 | 1 | 2 |
| 13000556 | 1 | 87 | 1 | 0 | 0 | 3 | 3 |
| 13000641 | 1 | 77 | 1 | 1 | 1 | 1 | 2 |
| 13000659 | 1 | 48 | 1 | 0 | 0 | 3 | 4 |
| 13000474 | 0 | 62 | 1 | 0 | 0 | 1 | 2 |
| 13000595 | 0 | 60 | 1 | 0 | 0 | 3 | 5 |
| 13600082 | 0 | 80 | 1 | 0 | 0 | 3 | 3 |
| 13000141 | 0 | 71 | 1 | 0 | 0 | 2 | 3 |
| 13000295 | 0 | 85 | 1 | 0 | 0 | 2 | 2 |
| 13000329 | 0 | 54 | 1 | 0 | 0 | 2 | 4 |
| 13000332 | 0 | 87 | 1 | 1 | 1 | 2 | 4 |
| 13000397 | 0 | 80 | 1 | 0 | 0 | 2 | 2 |
| 13000408 | 0 | 80 | 1 | 1 | 1 | 1 | N/A |
| 13000463 | 0 | 83 | 1 | 0 | 0 | 2 | N/A |
| 13000513 | 0 | 77 | 1 | 0 | 0 | 2 | 3 |
| 13000514 | 8 | 90 | 1 | 1 | 1 | 2 | 5 |
| 13000515 | 0 | 68 | 1 | 0 | 0 | 1 | 2 |
| 13000534 | 0 | 88 | 1 | 0 | 0 | 2 | 4 |
| 13000536 | 0 | 63 | 1 | 0 | 0 | 2 | 4 |
| 13000562 | 0 | 70 | 1 | 0 | 0 | 3 | 3 |
| 13000596 | 0 | 65 | 1 | 0 | 0 | 2 | 2 |
| 13000607 | 0 | 61 | 1 | 0 | 0 | 2 | 4 |
| 13000619 | 0 | 67 | 1 | 0 | 0 | 2 | 4 |
| 13000622 | 0 | 81 | 1 | 1 | 1 | 2 | 3 |
| 13000633 | 0 | 69 | 1 | 1 | 1 | 3 | 2 |
| 13000634 | 0 | 64 | 1 | 0 | 0 | 1 | 4 |
| 13000640 | 0 | 75 | 1 | 0 | 0 | 2 | N/A |
| 13000645 | 0 | 78 | 1 | 1 | 1 | 2 | N/A |
| 13006023 | 1 | 57 | 0 | 0 | 0 | N/A | N/A |
| 13000027 | 1 | 70 | 0 | 0 | 0 | N/A | N/A |
| 13000057 | 1 | 58 | 0 | 0 | 0 | N/A | N/A |
| 13000146 | 1 | 47 | 0 | 0 | 0 | N/A | N/A |
| 13000189 | 1 | 77 | 0 | 0 | 0 | N/A | N/A |
| 13000191 | 1 | 49 | 0 | 0 | 0 | N/A | NIA |
| 13000204 | 1 | 46 | 0 | 0 | 0 | N/A | N/A |
| 13000213 | 1 | 70 | 0 | 0 | 0 | N/A | N/A |
| 13000220 | 1 | 85 | 0 | 1 | 1 | N/A | N/A |
| 13000261 | 1 | 39 | 0 | 0 | 0 | N/A | N/A |
| 13000265 | 1 | 53 | 0 | 1 | 1 | N/A | N/A |
| 13060321 | 1 | 80 | 0 | 1 | 1 | N/A | N/A |
| 13006009 | 1 | 52 | 0 | 0 | 0 | N/A | N/A |
| 13000017 | 1 | 65 | 0 | 0 | 0 | N/A | N/A |
| 13000018 | 1 | 51 | 0 | 0 | 0 | N/A | N/A |
| 13000021 | 1 | 53 | 0 | 0 | 0 | N/A | N/A |
| 13000030 | 1 | 76 | 0 | 1 | 1 | N/A | N/A |
| 13000031 | 1 | 59 | 0 | 0 | 0 | N/A | N/A |
| 13000038 | 1 | 65 | 0 | 0 | 0 | N/A | N/A |
| 13000045 | 1 | 53 | 0 | 0 | 0 | N/A | N/A |
| 13000089 | 1 | 39 | 0 | 0 | 0 | N/A | N/A |
| 13000094 | 1 | 61 | 0 | 1 | 1 | N/A | N/A |
| 13000096 | 1 | 66 | 0 | 0 | 0 | N/A | N/A |
| 13000116 | 1 | 90 | 0 | 0 | 0 | N/A | N/A |
| 13000121 | 1 | 74 | 0 | 0 | 0 | N/A | N/A |
| 13000131 | 1 | 81 | 0 | 0 | 0 | N/A | N/A |
| 13000132 | 1 | 55 | 0 | 0 | 0 | N/A | N/A |
| 13000135 | 1 | 88 | 0 | 0 | 0 | N/A | N/A |
| 13000160 | 1 | 81 | 0 | 0 | 0 | N/A | N/A |
| 13000167 | 1 | 68 | 0 | 0 | 0 | N/A | N/A |
| 13000169 | 1 | 62 | 0 | 0 | 0 | N/A | N/A |
| 13000195 | 1 | 86 | 0 | 0 | 0 | N/A | N/A |
| 13000196 | 1 | 51 | 0 | 0 | 0 | N/A | N/A |
| 13000199 | 1 | 57 | 0 | 0 | 0 | N/A | N/A |
| 13000210 | 1 | 78 | 0 | 1 | 1 | N/A | N/A |
| 13000214 | 1 | 61 | 0 | 0 | 0 | N/A | N/A |
| 13000230 | 1 | 85 | 0 | 1 | 1 | N/A | N/A |
| 13000244 | 1 | 63 | 0 | 0 | 0 | N/A | N/A |
| 13000361 | 1 | 75 | 0 | 1 | 1 | N/A | N/A |
| 13000389 | 1 | 57 | 0 | 1 | 1 | N/A | N/A |
| 13000406 | 1 | 70 | 0 | 1 | 1 | N/A | N/A |
| 13000420 | 1 | 89 | 0 | 1 | 1 | N/A | N/A |
| 13000437 | 1 | 53 | 0 | 1 | 1 | N/A | N/A |
| 13000491 | 1 | 69 | 0 | 1 | 1 | N/A | N/A |
| 13000508 | 1 | 60 | 0 | 1 | 1 | N/A | N/A |
| 13000517 | 1 | 59 | 0 | 1 | 1 | N/A | N/A |
| 13000664 | 1 | 85 | 0 | 1 | 1 | N/A | N/A |
| 13000666 | 1 | 77 | 0 | 1 | 1 | N/A | N/A |
| 13000008 | 0 | 38 | 0 | 0 | 0 | N/A | N/A |
| 13000012 | 0 | 66 | 0 | 0 | 0 | N/A | N/A |
| 13000315 | 0 | 46 | 0 | 1 | 1 | N/A | N/A |
| 13000405 | 0 | 76 | 0 | 1 | 1 | N/A | N/A |
| 13000689 | 0 | 63 | 0 | 1 | 1 | N/A | N/A |
| 13000002 | 0 | 57 | 0 | 0 | 0 | N/A | N/A |
| 13000005 | 0 | 61 | 0 | 0 | 0 | N/A | N/A |
| 13000013 | 0 | 36 | 0 | 0 | 0 | N/A | N/A |
| 13000044 | 0 | 43 | 0 | 0 | 0 | N/A | N/A |
| 13000088 | 0 | 81 | 0 | 0 | 0 | N/A | N/A |
| 13000109 | 0 | 65 | 0 | 0 | 0 | N/A | N/A |
| 13000125 | 0 | 77 | 0 | 0 | 0 | N/A | N/A |
| 13000134 | 0 | 87 | 0 | 0 | 0 | N/A | N/A |
| 13000149 | 0 | 49 | 0 | 1 | 1 | N/A | N/A |
| 13000159 | 0 | 49 | 0 | 0 | 0 | N/A | N/A |
| 13000172 | 0 | 67 | 0 | 0 | 0 | N/A | N/A |
| 13000205 | 0 | 86 | 0 | 0 | 0 | N/A | N/A |
| 13000217 | 0 | 55 | 0 | 0 | 0 | N/A | N/A |
| 13000226 | 0 | 84 | 0 | 0 | 0 | N/A | N/A |
| 13006228 | 0 | 64 | 0 | 0 | 0 | N/A | N/A |
| 13600262 | 0 | 52 | 0 | 0 | 0 | N/A | N/A |
| 13000271 | 0 | 61 | 0 | 1 | 1 | N/A | N/A |
| 13000307 | 0 | 69 | 0 | 1 | 1 | N/A | N/A |
| 13000310 | 0 | 50 | 0 | 1 | 1 | N/A | N/A |
| 13000313 | 0 | 63 | 0 | 1 | 1 | N/A | N/A |
| 13000344 | 0 | 64 | 0 | 1 | 1 | N/A | N/A |
| 13000348 | 0 | 92 | 0 | 1 | 1 | N/A | N/A |
| 13000376 | 0 | 64 | 0 | 1 | 1 | N/A | N/A |
| 13000382 | 0 | 73 | 0 | 1 | 1 | N/A | N/A |
| 13000401 | 0 | 71 | 0 | 1 | 1 | N/A | N/A |
| 13000446 | 0 | 70 | 0 | 1 | 1 | N/A | N/A |
| 13000528 | 0 | 68 | 0 | 1 | 1 | N/A | N/A |
| 13000573 | 0 | 75 | 0 | 1 | 1 | N/A | N/A |
| 13000598 | 0 | 62 | 0 | 1 | 1 | N/A | N/A |
| 13000643 | 0 | 61 | 0 | 1 | 1 | N/A | N/A |
| 13000647 | 0 | 60 | 0 | 1 | 1 | N/A | N/A |
| 13000649 | 0 | 78 | 0 | 1 | 1 | N/A | N/A |
| 13000669 | 0 | 73 | 0 | 1 | 1 | N/A | N/A |
| 13000688 | 0 | 77 | 0 | 1 | 1 | N/A | N/A |
| 13000154 | 1 | 82 | 0 | 0 | 0 | N/A | N/A |
| 13000535 | 1 | 77 | 0 | 0 | 0 | N/A | N/A |

*For details on the different nomenclature and classification see Cubiella et al., Colorectal Dis. 2014 August; 16(8):O273-82; and Edge A, e al., AJCC Cancer Staging Manual, 7th edition. Berlin: Springer, 2010, N/A = not available or not applicable.

Sample Preparation and UPLC®-MS Metabolomics Analysis

An UPLC-time-of-flight (TOF)-MS based platform was used to analyse chloroform/methanol extract. Glycerolipids, cholesteryl esters, sphingolipids, primary fatty amides and glycerophospholipids were among the ion features identified. The metabolite extraction procedure was as follows:

15 milligrams of lyophilized stool samples were mixed with 45 μL sodium chloride (50 mM) and 450 μL chloroform/methanol (30:1) in 1.5 mL microtubes at room temperature. The extraction solvent was spiked with compounds not detected in unspiked human stool samples [SM(d18:1/16:0), PE(17:0/17:0), PC(19:0/19:0), TAG(13:0/13:0/13:0), Cer(d18:1/17:0) and ChoE(12:0)]. After brief vortex mixing, the samples were incubated for 1 hour at −20° C. After centrifugation at 16,000×g for 15 minutes, 35 μL of the lower organic phase was collected and the solvent removed. The dried extracts were then reconstituted in 1000 μL acetronitrile/isopropanol (1:1), centrifuged (16,000×g for 5 minutes), and transferred to vials for UPLC®-MS analysis on an Acquity-Xevo G2 QTof system (Waters Corp., Milford, Mass.). Samples were randomly divided into three batches, which contained a maximum of 78 samples. Chromatographic method and mass spectrometric detection conditions were described by Barr et al. (J Proteome Res, 2012, 11, 2521-2532).

FOB Test.

FOB was determined using the automated OC-SENSOR MICRO analyser (Eiken Chemical Co., Ltd, Tokyo, Japan) according to the instructions of the manufacturer. The test method is an immunologic test based on a latex agglutination reaction. A latex reagent is prepared by sensitizing anti-human HbA0 antibodies to polystyrene latex particles. When this reagent is mixed with the sample, the anti-human HbA₀ antibodies, which were sensitized to latex, react with the hemoglobin in the sample, and the latex aggregate is formed in the latex agglutination reaction. The change in absorbance per unit time resulting from the latex agglutination reaction is proportional to the concentration of hemoglobin in the sample. A dose response curve of the absorbance unit (OD) vs. concentration is generated using the results obtained from the calibrators. The concentration of hemoglobin in the patient sample is determined from this curve.

Data Pre-Processing

Data pre-processing was processed using the TargetLynx application manager for MassLynx 4.1 (Waters Corp). A total of 133 UPLC-MS features were analysed, all of them identified prior to the analysis. Peak detection and noise reduction were performed as previously described (J Proteome Res, 2012, 11, 2521-2532). Intra- and inter-batch normalization process was based on multiple internal standards and pool calibration samples approach described by Martinez-Arranz et al. (J Proteomics. 2015, 8, 127(Pt B):275-88.).

Data Analysis

Univariate was assessed by the Shapiro-Wilk test. Univariate statistical analyses were also performed calculating group percentage changes and the analysis of variance (ANOVA) and Tukey's HSD (Honestly Significance Difference) post hoc test for the comparison among the different groups: CRC, AD and control (C). Wilcoxon signed-rank test/»-values were calculated for the comparison between cases (AD and CRC) and controls (healthy) groups, as well as for the comparisons CRC and control, CRC and AD and between AD and control groups. The threshold for significance was p<0.05.

A logistic regression (LR) was performed to identify a predictive signature capable of distinguishing between cases and control groups. LR is a common used technique for data classification and dimensionality reduction. A forward stepwise method was selected as variable selection approach, where the analysis started with an empty model and variables were added one at a time as long as these additions are worthy. Once a variable was included, the model was evaluated to ensure its discrimination capability. This process finished when no more variables could be added. All samples were randomly divided into estimation (90% of all subjects; n=116) and validation (10% of all subjects; n=13) groups, having both cohorts an equal proportional representation of individuals belonging to cases and control groups. Receiver operating characteristic (ROC) curve analysis was used to assess its discriminatory power. Overall diagnostic accuracy for a given two-class comparison was given by the area under the ROC curve (AUC) with its associated standard error. Sensitivity, specificity, positive predictive value, and negative predictive value were calculated. All calculations were performed using statistical software package R v.3.1.1 (R Development Core Team, 2011; http://cran.r-project.org) with caret, caTools and receiver operating characteristic R (ROCR) packages to produce ROC curves and AUC estimate; and MASS package to generate the LR. Additionally, SIMCA-P+ 12.0.1 (Umetrics AB, Umea, Sweden) was used for PCA and OPLS multivariate data analysis.

Results

A test is considered to be a suitable discriminator if the AUC is from 0.6 to 0.75, to have a high discrimination if the AUC is from 0.75 to 0.9 and to be an excellent discriminator if the AUC is from 0.9 to 1.

I. First Biomarker Signature

Using a regression model a first group of 4 biomarkers was identified as biomarker signature. ChoE(18:2) was found to be the biomarker presenting the best AUC to distinguish between healthy controls (HC) and disease (CRC+AD) population.

1) Reference Values (Average After Data Processing & Normalization)

TABLE 3

| | Average (±Standard Deviation) | | | |
|---|---|---|---|---|
| | Control | Cases (CRC − AD) | CRC | AD |
| ChoE(18:0) | 0.158(±0.205) | 0.161(±0.237) | 0.133(±0.127) | 0.190(±0.313) |
| ChoE(18:2) | 0.016(±0.016) | 0.063(±0.121) | 0.103(±0.156) | 0.023(±0.044) |
| SM(d18:0/14:0) | 0.225(±0.180) | 0.410(±0.328) | 0.393(±0.268) | 0.432(±0.410) |
| TG(54:3) | 2.541(±5.596) | 2.013(±7.544) | 3.476(±10.814) | 0.699(±0.977) |

2) Regression Model

TABLE 4

| | Control vs Disease (CRC + AD) | | | Control vs CRC | | | CRC vs AD | | |
|---|---|---|---|---|---|---|---|---|---|
| | AUC | Sensitivity | Specificity | AUC | Sensitivity | Specificity | AUC | Sensitivity | Specificity |
| ChoE(18:0) | 0.567 | 0.500 | 0.733 | 0.275 | 0.750 | 0.200 | 0.304 | 0.375 | 0.429 |
| ChoE(18:2) | 0.743 | 0.800 | 0.667 | 0.988 | 1.000 | 0.900 | 1.000 | 1.000 | 1.000 |
| SM(d18:0/14:0) | 0.453 | 0.500 | 0.533 | 0.525 | 0.625 | 0.500 | 0.518 | 0.625 | 0.714 |
| TG(54:3) | 0.467 | 0.500 | 0.733 | 0.450 | 0.750 | 0.500 | 0.446 | 0.875 | 0.286 |
| All together | 0.713 | 1.000 | 0.533 | 0.875 | 0.750 | 1.000 | 0.964 | 0.875 | 1.000 |

In combination with FOB

| | | | | | | | | | |
|---|---|---|---|---|---|---|---|---|---|
| ChoE(18:0) + FOB | 0.907 | 0.900 | 0.867 | 0.963 | 0.875 | 0.900 | 0.411 | 0.750 | 0.429 |
| ChoE(18:2) + FOB | 0.913 | 0.900 | 0.867 | 0.963 | 0.875 | 0.900 | 0.911 | 0.875 | 1.000 |
| SM(d18:0114:0) + FOB | 0.867 | 0.900 | 0.800 | 0.800 | 0.750 | 1.000 | 0.536 | 0.500 | 0.714 |
| TG(54:3) + FOB | 0.800 | 0.900 | 0.800 | 0.750 | 0.750 | 0.900 | 0.429 | 0.625 | 0.429 |
| All together + FOB | 0.887 | 0.900 | 0.800 | 0.900 | 0.875 | 0.900 | 0.839 | 0.750 | 1.000 |

I. Second Biomarker Signature

In order to avoid the population effects when subsetting the data into train and test, we generated a 10,000 times iteration for both subsetting and model construction. We estimated the median of the Area Under the Curve (AUC) of the 10,000 iterations and we added metabolites one by one to the model, in case that addition resulted in a better AUC than the model's one. All statistical analysis was performed using R software v3.3.2 (R Development Core Team, 2016; http://cran.r-project.org) with stats, caret, ggfortify, ROCR and modEvA packages. PCA study and plots were generated with SIMCA-P v12.0.1.0, by Umetrics AB.

Further to this iteration process, a second biomarker signature was obtained which is more resistant to variations in the population. This second signature has ChoE(18:2) in common with the first signature, thus, strengthens the diagnostic value of this metabolic marker.

1) Reference Values (Average After Data Processing & Normalization)

TABLE 5

| | Average (±Standard Deviation) | | | |
|---|---|---|---|---|
| | Control | Cases (CRC − AD) | CRC | AD |
| ChoE(18:2) | 0.016(±0.016) | 0.063(±0.121) | 0.103(±0.156) | 0.023(±0.044) |
| ChoE(18:1) | 0.093(±0.102) | 0.180(±0.394) | 0.196(±0.217) | 0.163(±0.514) |
| ChoE(20:4) | 0.015(±0.032) | 0.070(±0.162) | 0.119(±0.215) | 0.018(±0.024) |
| PE(16:0/18:1) | 1.528(±2.295) | 2.564(±3.244) | 2.866(±3.447) | 2.263(±3.041) |
| SM(d18:1/23:0) | 0.008(±0.010) | 0.018(±0.045) | 0.017(±0.027) | 0.019(±0.066) |
| SM(42:3) | 0.002(±0.003) | 0.049(±0.273) | 0.026(±0.047) | 0.077(±0.409) |
| TG(54:1) | 4.294(±12.988) | 2.147(±6.372) | 3.499(±8.623) | 0.795(±2.037) |

TABLE 6

| | Control vs Disease (CRC + AD) | | | Control vs CRC | | | CRC vs AD | | |
|---|---|---|---|---|---|---|---|---|---|
| | AUC | Sensitivity | Specificity | AUC | Sensitivity | Specificity | AUC | Sensitivity | Specificity |
| ChoE (18:2) | 0.688 | 0.700 | 0.688 | 0.812 | 0.800 | 0.750 | 0.766 | 0.750 | 0.750 |
| ChoE (18:1) | 0.603 | 0.600 | 0.625 | 0.650 | 0.700 | 0.625 | 0.609 | 0.625 | 0.750 |
| ChoE (20:4) | 0.712 | 0.800 | 0.625 | 0.650 | 0.700 | 0.625 | 0.789 | 0.750 | 0.750 |
| PE (16:0/18:1) | 0.644 | 0.700 | 0.625 | 0.700 | 0.700 | 0.750 | 0.609 | 0.750 | 0.750 |
| SM (d18:1/23:0) | 0.619 | 0.600 | 0.625 | 0.712 | 0.700 | 0.750 | 0.711 | 0.750 | 0.750 |
| SM (42:3) | 0.659 | 0.667 | 0.636 | 0.771 | 0.833 | 0.714 | 0.750 | 0.714 | 0.833 |
| TG (54:1) | 0.581 | 0.600 | 0.625 | 0.538 | 0.600 | 0.625 | 0.574 | 0.625 | 0.625 |
| All metabs | 0.821 | 0.833 | 0.833 | 0.929 | 0.889 | 0.875 | 0.833 | 0.750 | 1.000 |

TABLE 7

| | Control vs Disease (CRC + AD) | | | Control vs CRC | | | CRC vs AD | | |
|---|---|---|---|---|---|---|---|---|---|
| | AUC | Sensitivity | Specificity | AUC | Sensitivity | Specificity | AUC | Sensitivity | Specificity |
| ChoE (18:2) + FOB | 0.797 | 0.900 | 0.750 | 0.925 | 0.900 | 0.875 | 0.781 | 0.750 | 0.750 |
| ChoE (18:1) + FOB | 0.775 | 0.800 | 0.688 | 0.875 | 0.900 | 0.875 | 0.688 | 0.875 | 0.625 |
| ChoE (20:4) + FOB | 0.791 | 0.800 | 0.750 | 0.875 | 0.900 | 0.875 | 0.797 | 0.750 | 0.750 |
| PE (16:0/18:1 + FOB | 0.800 | 0.800 | 0.750 | 0.862 | 0.800 | 0.750 | 0.703 | 0.750 | 0.750 |
| SM (d18:1/23:0) + FOB | 0.788 | 0.800 | 0.750 | 0.894 | 0.900 | 0.875 | 0.766 | 0.750 | 0.750 |
| SM (42:3) + FOB | 0.822 | 0.857 | 0.769 | 0.896 | 0.875 | 0.875 | 0.771 | 0.714 | 0.857 |
| TG (54:1) + FOB | 0.800 | 0.800 | 0.813 | 0.838 | 0.900 | 0.875 | 0.633 | 0.750 | 0.625 |
| All metabs + FOB | 0.885 | 0.875 | 0.846 | 0.929 | 1.000 | 0.875 | 0.857 | 0.857 | 0.857 |

1. In vitro method for the screening, diagnosis or monitoring of colorectal cancer and/or advanced colorectal adenoma in a subject, said method comprising the following steps:
a) determining the levels of the metabolic marker cholesteryl ester (ChoE) (18:2) in a feces sample isolated from said subject;
b) comparing the levels in said feces sample with a reference value;
wherein an increase of ChoE(18:2) levels in the subject sample with regard to said reference value is indicative of colorectal cancer and/or advanced adenoma.

2. The method according to claim 1, wherein said subject is suspected of having or at an increased risk of having colorectal cancer and/or advanced adenoma.

3. The method according to any of claim 1 or 2, for the screening, diagnosis or monitoring of colorectal cancer, preferably wherein said colorectal cancer is adenocarcinoma.

4. The method according to any of claims 1 to 3, wherein said reference value is obtained from a subject or group of subjects not having a gastrointestinal disease.

5. The method according to any of claims 1 to 3, wherein said reference value is obtained from a subject or group of subjects having advanced adenoma.

6. The method according to any of claims 1 to 5 wherein said method further comprises the determination of the levels of at least one further metabolic marker, preferably all, selected from the group consisting of ChoE(18:0), triacylglycerol (TG) (54:3), sphingomyelin (SM) (d18:0/14:0), ChoE(18:1), ChoE(20:4), phosphatidylethanolamine (PE) (16:0/18:1), SM(d18:1/23:0), SM(42:3) and TG(54:1); and comparing the levels in said feces sample for said metabolic marker with a reference value.

7. The method according to claim 6, wherein said further metabolic marker is selected from the group consisting of ChoE(18:1), ChoE(20:4), PE(16:0/18:1), SM(d18:1/23:0), SM(42:3) and TG(54:1).

8. The method according to any of claims 1 to 7, wherein the determination of the levels of said metabolic marker is conducted by a mass spectrometry-based method, preferably by ultra-performance liquid chromatography/time-of-flight mass spectrometry.

9. The method according to any of claims 1 to 8, wherein said method further comprises conducting a fecal occult blood test.

10. The method according to any of claims 1 to 9, wherein said method further comprises conducting an exploratory test selected from the group consisting of colonoscopy, flexible sigmoidoscopy, double-contrast barium enema and computed tomography (CT) colonography.

11. The method according to any of claims 1 to 10, wherein said subject is a human subject.

12. A method according to any of claims 1 to 11, wherein said method further comprises storing the method results in a data carrier, preferably wherein said data carrier is a computer readable medium.

13. A computer implemented method, wherein the method is as defined in any of claims 1 to 11.

14. Use of a kit for the screening, diagnosis or monitoring of colorectal cancer and/or advanced colorectal adenoma in a subject, said kit comprising:
a) labelled and/or unlabeled ChoE(18:2), and optionally at least one labelled and/or unlabeled further metabolic marker, preferably all, selected from the list consisting of ChoE(18:0), TG(54:3), SM(d18:0/14:0), ChoE(18:1), ChoE(20:4), PE(16:0/18:1), SM(d18:1/23:0), SM(42:3) and TG(54:1), in a feces sample;
b) optionally, instructions for the use of reagent(s) in a) in determining the levels of ChoE(18:2), and optionally of at least said one further metabolic marker, in a feces sample.
